(19) United States
(12) Patent Application Publication
(10) Pub. No.: US 2016/0296171 A1
(43) Pub. Date: Oct. 13, 2016

(54) A PATIENT MONITORING SYSTEM

(71) Applicant: CARDIOLOGIC INNOVATIONS LTD, Neve Ilan (IL)

(72) Inventors: Oren DRORI, BINYAMINA (IL); Shimon ARAD (ABBOUD), TEL AVIV (IL); Yoav TIKOCHINSKY, TEL-AVIV (IL); Haim KRIEF, HADERA (IL); Shay FEITELZON, MOSHAV KLACHIM (IL)

(73) Assignee: CARDIOLOGIC INNOVATIONS LTD, NEVE ILAN (IL)

(21) Appl. No.: 15/100,349

(22) PCT Filed: Nov. 30, 2014
(86) PCT No.: PCT/IL2014/051037

§ 371 Date: May 31, 2016

Related U.S. Application Data
(60) Provisional application No. 61/910,359, filed on Dec. 1, 2013.

(51) Int. Cl. A61B 5/00; A61B 5/024; A61B 5/107; A61B 5/08; A61B 5/021; A61B 5/053; A61B 5/0402

(52) U.S. Cl. CPC A61B 5/6841; A61B 5/6823; A61B 5/6846; A61B 5/0536; A61B 5/024; A61B 5/0402; A61B 5/0816; A61B 5/021; A61B 5/4839; A61B 5/742; A61B 5/1072; A61B 5/6842; A61B 2560/0223

(57) ABSTRACT

A patient monitoring system comprising: at least two pairs of electrodes in contact with the patient, a controller and a patient measurement system. At least one pair of electrodes passes a known current through a patient's chest. The voltage difference at another position on the patient induced by the known current is detected by a second at least one pair of electrodes and measured by a voltage measuring unit. From the voltage differences measured between the at least one second pair of the electrodes, the controller determines at least one biological parameter in at least one portion of the patient's chest. The patient measurement system ensures correct placement of the pairs of electrodes on the patient's chest, such that the electrodes are consistently placeable in the same positions on the patient's chest.

### FIELD OF THE INVENTION

The present invention generally pertains to a system and method for monitoring and evaluating biophysical measurements in the body. In particular, the disclosure relates to a system where electrodes are placed on the skin in a predetermined pattern to enable measurement of biophysical parameters of the thorax.

### BACKGROUND OF THE INVENTION

Biophysical parameters of the thoracic region of a patient can be measured by using pairs of electrodes to inject current into the thoracic region, and other pairs of electrodes to measure the potential difference generated by the injected currents. In some systems, the electrodes are implanted. However, there are disadvantages to using implanted electrodes, including the surgery needed to implant them in the first place, the possibility of infection at the point where the electrode leads exit the body, the possibility of reactions between the body and the implanted electrodes or their leads, and the need for a second surgical operation to remove the electrodes when they are no longer needed.

Therefore, it is preferable, in most cases, to place the electrodes in close contact with the skin of the patient. However, electrical thoracic scans are sensitive to the location of the electrodes and placing these electrodes securely and in the correct locations is a delicate task that requires time, as well as extensive training. Thus, there is a need for a device that enables the correct placement of the electrodes in the correct locations without delicate user instruction (i.e., being user-agnostic).

Among the many applications of electrical thoracic scans is the detection of pulmonary edema. Pulmonary edema is characterized by a buildup of extracellular fluid in the lungs. It leads to impaired gas exchange and may cause respiratory failure. The condition may have various causes. Pulmonary edema may be cardiogenic, caused by improper heart function, e.g., congestive heart failure (CHF). As such, a reduction in extracellular fluid (e.g., in the lungs) in CHF patients typically indicates an improvement in heart performance. Pulmonary edema may also be caused by an injury to the lungs themselves.

Conventional methods of monitoring pulmonary edema in patients either require expensive equipment and trained personnel (e.g. measuring pulmonary artery and central venous pressure with catheters, measuring blood flow through the mitral annulus and pulmonary veins with doppler echocardiography) or are not very accurate (e.g. monitoring changes in body weight, observing neck vein distension, measuring ankle dimensions as a proxy for congestion caused by heart failure).

Pulmonary edema can be measured reliably using electrical thoracic scans. One such electrical thoracic scan is electrical impedance measurement, and another is electrical impedance tomography (EIT). Electrical impedance measurements of the torso have been shown to correlate with the level of retained body water, for example extracellular water in the lungs. Electrical impedance tomography of the torso may be used to monitor the presence and/or severity of pulmonary edema with a high level of accuracy, with less invasiveness to the patient and at lower cost. See, e.g., U.S. Pat. No. 7,096,061 and U.S. patent publication 20120150050.

It is therefore a long felt need to provide a system that accurately measures pulmonary edema which is not invasive, which does not require intensive training and that does not take large amounts of setup time.

### SUMMARY OF THE INVENTION

It is an object of the present invention to disclose a system for measuring at least one biological parameter of the chest using a patient monitoring system employing electrodes.

It is another object of the present invention to disclose a patient monitoring system comprising:
a. at least one first pair of electrodes wired to each other, adapted to pass known current through said patient's chest;
b. a voltage measuring unit capable of measuring a voltage difference between at least one second pair of electrodes when said first pair of electrodes is passing said known current through said portion of said patient through said first pair of electrodes, when said first and said second pair of electrodes are in contact with said patient;
c. a controller capable of determining at least one biological parameter in at least one portion of said patient's chest, using one or more said voltage differences measured between said at least one second pair of the electrodes; and
d. a patient measurement system;
   wherein said patient measurement system is adapted to ensure correct placement of said at least one first pair of electrodes and said at least one second pair of electrodes on said patient's chest, such that said at least one first pair of electrodes and said at least one second pair of electrodes are consistently placeable in the same position on said patient's chest.

It is another object of the present invention to disclose the system, wherein electrical contact of said electrodes with said patient is provided by a member of a group consisting of: at least one of said electrodes is operable to contact the skin surface of a patient, at least one of said electrodes is implantable in said patient, and any combination thereof.

It is another object of the present invention to disclose the system, wherein the electrodes are placed in positions selected from a group consisting of: at predetermined fractions of the circumference, starting from at least one predetermined point; at predetermined distances from at least one starting point; and any combination thereof.

It is another object of the present invention to disclose the system, wherein said predetermined starting point is selected from a group consisting of: the sternum, the front of the armpit, the back of the armpit, the backbone, and any combination thereof.

It is another object of the present invention to disclose the system, wherein said electrodes are either equally spaced on said patient's chest, or unequally spaced on said patient's chest.

It is another object of the present invention to disclose the system, wherein said electrodes are placed in a manner selected from a group consisting of: selecting a fixed point on said patient's chest and placing the electrodes at defined angular positions from said fixed point around said chest; placing the electrodes in an equally spaced manner; placing the electrodes symmetrically with respect to the sagittal plane of said chest; and placing the electrodes symmetrically with respect to the coronal plane of said chest; and any combination thereof.

It is another object of the present invention to disclose the system, wherein said patient's chest is said patient's thorax.

It is another object of the present invention to disclose the system, wherein said biological parameter is selected from a group consisting of resistance, derived resistivity, conductance, derived conductivity, derived lung fluid volume, cardiac rate, ECG, respiratory rate, respiratory pattern, blood pressure, and any combination thereof.

It is another object of the present invention to disclose the system, wherein said lung fluid volume is determined from said conductivity.

It is another object of the present invention to disclose the system, wherein said lung fluid is extracellular fluid.

It is another object of the present invention to disclose the system, wherein said monitoring system is configured for a monitoring procedure selected from a group consisting of electrocardiography and body surface mapping.

It is another object of the present invention to disclose the system, wherein said system is configured to monitor at least one selected from a group consisting of: bio-impedance; impedance cardiography; intra-thoracic impedance, phase-delay measurement and any combination thereof.

It is another object of the present invention to disclose the system, wherein the system is configured for a monitoring procedure selected from a group consisting of: (a) electrical impedance tomography (EIT), wherein said voltage measuring instrument is configured to measure the voltage in a plurality of pairs of electrodes and said controller is configured to calculate said biological parameter based on said measured voltages, said biological parameter being conductivity in a plurality of voxels in the thorax; and (b) parametric EIT (pEIT), wherein said voltage measuring instrument is configured to measure voltages in a plurality of pairs of electrodes and said controller is configured to calculate said biological parameter based on said measured voltages, said biological parameter being at least one of a group consisting of conductivity, resistivity, conductance and resistance of at least one organ in the thorax.

It is another object of the present invention to disclose the system, wherein said organ is a heart or a lung.

It is another object of the present invention to disclose the system, wherein said system additionally comprises means adapted to deliver at least one drug to said patient.

It is another object of the present invention to disclose the system, wherein said drug delivery means is a computer-controllable drug delivery device, wherein the drug is delivered by a method selected from a group consisting of injection, providing pills and providing a drinkable liquid.

It is another object of the present invention to disclose the system, wherein said drug delivery means is adapted to titrate said at least one drug to said patient, such that said system is adapted to provide closed-loop monitoring of said patient.

It is another object of the present invention to disclose the system, wherein said system further comprises a screen adapted to display at least one said at least one biological parameter.

It is another object of the present invention to disclose the system, wherein said controller further comprises a database adapted to store at least one said at least one biological parameter.

It is another object of the present invention to disclose the system, wherein said system further comprises means to enable a user to enter a comment, said comment storable in said database.

It is another object of the present invention to disclose the system, wherein said means to enable a user to enter a comment is selected from a group consisting of a keyboard, a touchscreen, voice commands and any combination thereof.

It is another object of the present invention to disclose the system, wherein said comment is displayable on said display unit.

It is another object of the present invention to disclose the system, wherein said screen is adapted to display, for each said comment, at least one indication that said comment has been entered into said system.

It is another object of the present invention to disclose the system, wherein said screen is adapted to indicate, for each said comment, the time each said comment was entered.

It is another object of the present invention to disclose the system, wherein said screen comprises a touchscreen.

It is another object of the present invention to disclose the system, wherein touching said at least one said indication displays said comment.

It is another object of the present invention to disclose the system, wherein said electrodes are either commercially available electrodes or proprietary electrodes.

It is another object of the present invention to disclose the system, wherein the size of said patient's thoracic region is selected from a group consisting of: its width, its depth (front-to-back), its circumference, its perimeter length, its diameter, its radius, the length of an axis, its cross-sectional area, its surface area, its volume, and any combination thereof and any combination thereof.

It is another object of the present invention to disclose the system, wherein said patient measurement system comprises at least one mechanism for measuring the size of at least a portion of said thoracic region.

It is another object of the present invention to disclose the system, wherein said mechanism for measuring the size of at least a portion of said thoracic region is selected from a group consisting of an anthropometer, a measuring tape and any combination thereof

It is another object of the present invention to disclose the system, wherein said patient measurement system comprises at least one mechanism for reproducibly specifying at least one position on said thoracic region.

It is another object of the present invention to disclose the system, wherein said mechanism for reproducibly specifying at least one position on said thoracic region comprises a placement accessory, said placement accessory adapted to ergonomically guide correct positioning of said electrodes and thereby to enable consistent and repeatable placement of said electrodes at a predetermined position on the patient's thoracic region.

It is another object of the present invention to disclose the system, wherein said placement accessory is L-shaped.

It is another object of the present invention to disclose the system, wherein said placement accessory is disposable.

It is another object of the present invention to disclose the system, wherein said placement accessory comprises a slit of predetermined shape, said slit adapted to enable the surface of said patient's thoracic region to be marked.

It is another object of the present invention to disclose the system, wherein said patient measurement system comprises at least one mechanism for marking on the surface of said thoracic region in at least one predetermined position.

It is another object of the present invention to disclose the system, wherein said mechanism for marking on the surface of said thoracic region comprises a pen, a pencil, a marking pen, an IR laser marker, a temporary tattoo, a sticker, a frangible ink cartridge and any combination thereof.

It is another object of the present invention to disclose the system, wherein at least one of said temporary tattoo, said sticker and said frangible ink cartridge is mounted on said placement accessory.

It is another object of the present invention to disclose the system, wherein said placement accessory comprises said mechanism for marking on the surface of said thoracic region.

It is another object of the present invention to disclose the system, further comprising a power supply adapted to power said screen, said controller, and to provide said current to said electrodes.

It is another object of the present invention to disclose the system, wherein said measurement system is comprised in a first kit adapted to be provided as a unit.

It is another object of the present invention to disclose the system, wherein a second kit adapted to be provided as a unit comprises at least one of a group consisting of: said mechanism for measuring the size of at least a portion of said chest, said mechanism for reproducibly specifying at least one position on said chest, and said mechanism for marking on the surface of said chest each said at least one position.

It is another object of the present invention to disclose the system, wherein said biological parameter is determined from a calibrated voltage difference.

It is another object of the present invention to disclose the system, wherein said calibration is selected from a group consisting of: calibration to the size of the patient, calibration to the patient's breathing cycle, and any combination thereof.

It is another object of the present invention to disclose the system, wherein said size calibration is a linear calibration.

It is another object of the present invention to disclose the system, wherein said size calibration is calculated from V_{c}=Vₘ−a(Pₘ−P_{c}), where a is a predetermined constant, Pₘ is the measured patient size and P_{c} is a predetermined standard cross-section size.

It is another object of the present invention to disclose the system, wherein said measured patient size is selected from a group consisting of: width of a predetermined portion of the body, circumference of a predetermined portion of the body, area of a predetermined portion of the body, thickness of a predetermined portion of the body, and any combination thereof.

It is another object of the present invention to disclose the system, wherein said portion of the body is selected from a group consisting of: a predetermined portion of the thorax, a predetermined portion of the height, a predetermined portion of the abdomen, and any combination thereof.

It is another object of the present invention to disclose the system, wherein said calibration to the patient's breathing cycle is selected from a group consisting of: calibration to a single breathing cycle, calibration to a predetermined portion of a breathing cycle, calibration to a plurality of breathing cycles, and any combination thereof.

It is another object of the present invention to disclose the system, wherein said calibration to a single breathing cycle consists of averaging a plurality of voltage difference measurements, said voltage difference measurements taken during a single breathing cycle.

It is another object of the present invention to disclose the system, wherein said average of said voltage difference measurements for a single breathing cycle is selected from a group consisting of: the mean of the voltage difference measurements, the median of the voltage difference measurements and the mode of the voltage difference measurements.

It is another object of the present invention to disclose the system, wherein said predetermined portion of a breathing cycle is selected from a group consisting of: at least a portion of an inspiration, at least a portion of an exhalation, the beginning of an inspiration, the beginning of an exhalation, the end of an inspiration, the end of an exhalation, and any combination thereof.

It is another object of the present invention to disclose the system, wherein said calibration to said predetermined portion of a breathing cycle consists of averaging a plurality of voltage difference measurements, said voltage difference measurements taken either during a single breathing cycle or during a plurality of breathing cycles.

It is another object of the present invention to disclose the system, wherein said average of said voltage difference measurements for said predetermined portion of a breathing cycle is selected from a group consisting of: the mean of the voltage difference measurements, the median of the voltage difference measurements and the mode of the voltage difference measurements.

It is another object of the present invention to disclose the system, wherein said calibration to a plurality of breathing cycles consists of averaging a plurality of voltage difference measurements, each said voltage difference measurement being the minimum voltage difference measured during a single breathing cycle.

It is another object of the present invention to disclose the system, wherein said average of said voltage difference measurements for a plurality of breathing cycles is selected from a group consisting of: the mean of the voltage difference measurements, the median of the voltage difference measurements and the mode of the voltage difference measurements.

It is another object of the present invention to disclose a method for monitoring a patient comprising steps of:

a. providing a system for monitoring the lung fluid volume of patients, comprising:
i. at least one first pair of electrodes wired to each other, adapted to pass known current through said patient's chest;
ii. a voltage measuring unit capable of measuring a voltage difference between at least one second pair of electrodes when said first pair of electrodes is passing said known current through said portion of said patient through said first pair of electrodes, when said first and said second pair of electrodes are in contact with said patient;
iii. a controller capable of determining at least one biological parameter in at least one portion of said patient's chest, using one or more said voltage differences measured between said at least one second pair of the electrodes; and
iv. a patient measurement system;
   b. measuring said patient with said patient measurement system;
   c. placing said electrodes in electrical contact with said patient as indicated by said patient measurement system, thereby ensuring correct placement of said at least one first pair of electrodes and said at least one second pair of electrodes on said patient's chest, such that said at least one first pair of electrodes and said at least one second pair of electrodes are consistently placeable in the same position on said patient's chest;
   d. measuring, with said voltage measuring unit, at least one voltage difference across at least one pair of said at least one second pair of electrodes;
   e. determining, with said controller, using at least one of said at least one voltage differences, at least one of said at least one biological parameters in at least one portion of said patient's chest.

It is another object of the present invention to disclose the method, wherein said steps of placing said electrodes in electrical contact with said patient are selected from a group consisting of: steps of contacting the skin surface of a patient with at least one said electrode, steps of using at least one said electrode implanted in said patient, and any combination thereof.

It is another object of the present invention to disclose the method, additionally comprising steps of spacing said electrodes either equally spaced on said patient's chest, or unequally spaced on said patient's chest.

It is another object of the present invention to disclose the method, additionally comprising steps of placing the electrodes in positions selected from a group consisting of: at predetermined fractions of the circumference, starting from at least one predetermined point; at predetermined distances from at least one starting point; and any combination thereof.

It is another object of the present invention to disclose the method, additionally comprising steps of selecting said predetermined starting point is selected from a group consisting of: the sternum, the front of the armpit, the back of the armpit, the backbone, and any combination thereof.

It is another object of the present invention to disclose the method, additionally comprising steps of placing said electrodes on said patient's chest in a manner selected from a group consisting of: selecting a fixed point on said patient's chest and placing the electrodes at defined angular positions from said fixed point around said chest; placing the electrodes in an equally spaced manner; placing the electrodes symmetrically with respect to the sagittal plane of said chest; placing the electrodes symmetrically with respect to the coronal plane of said chest; and any combination thereof.

It is another object of the present invention to disclose the method, wherein said patient's chest is said patient's thorax.

It is another object of the present invention to disclose the method, additionally comprising steps of selecting said biological parameter from a group consisting of resistance, derived resistivity, conductance, derived conductivity, derived lung fluid volume, cardiac rate, ECG, respiratory rate, respiratory pattern, blood pressure, and any combination thereof.

It is another object of the present invention to disclose the method, additionally comprising steps of determining said lung fluid volume from said conductivity.

It is another object of the present invention to disclose the method, wherein said lung fluid is extracellular fluid.

It is another object of the present invention to disclose the method, additionally comprising steps of configuring said monitoring system for a monitoring procedure selected from a group consisting of electrocardiography and body surface mapping.

It is another object of the present invention to disclose the method, additionally comprising steps of configuring said system to monitor at least one selected from a group consisting of: bio-impedance; impedance cardiography; intra-thoracic impedance, phase-delay measurement and any combination thereof.

It is another object of the present invention to disclose the method, additionally comprising steps of configuring the system for a monitoring procedure selected from a group consisting of: (a) electrical impedance tomography (EIT), wherein said voltage measuring instrument is configured to measure the voltage in a plurality of pairs of electrodes and said controller is configured to calculate said biological parameter based on said measured voltages, said biological parameter being conductivity in a plurality of voxels in the thorax; and (b) parametric EIT (pEIT), wherein said voltage measuring instrument is configured to measure voltages in a plurality of pairs of electrodes and said controller is configured to calculate said biological parameter based on said measured voltages, said biological parameter being at least one of a group consisting of conductivity, resistivity, conductance and resistance of at least one organ in the thorax.

It is another object of the present invention to disclose the method, additionally comprising steps of selecting said organ from a group consisting of a heart and a lung.

It is another object of the present invention to disclose the method, additionally comprising steps of providing said system with means adapted to deliver at least one drug to said patient.

It is another object of the present invention to disclose the method, additionally comprising steps of providing said drug delivery means as a computer-controllable drug delivery device, wherein the drug is delivered by a method selected from a group consisting of injection, providing pills and providing a drinkable liquid.

It is another object of the present invention to disclose the method, additionally comprising steps of providing closed-loop monitoring of said patient by using said drug delivery means to titrate said at least one drug to said patient.

It is another object of the present invention to disclose the method, additionally comprising steps of providing a screen adapted to display at least one said at least one biological parameter.

It is another object of the present invention to disclose the method, additionally comprising steps of providing said controller with a database adapted to store at least one said at least one biological parameter.

It is another object of the present invention to disclose the method, additionally comprising steps of providing means enabling a user to enter a comment, said comment storable in said database.

It is another object of the present invention to disclose the method, additionally comprising steps of selecting said means to enable a user to enter a comment from a group consisting of a keyboard, a touchscreen, voice commands and any combination thereof.

It is another object of the present invention to disclose the method, additionally comprising steps of displaying said comment on said display unit.

It is another object of the present invention to disclose the method, additionally comprising steps of, for each said comment, displaying on said screen at least one indication that said comment has been entered into said system.

It is another object of the present invention to disclose the method, additionally comprising steps of indicating on said screen, for each said comment, the time each said comment was entered.

It is another object of the present invention to disclose the method, additionally comprising steps of providing said screen as a touchscreen.

It is another object of the present invention to disclose the method, additionally comprising steps of displaying said comment by touching said at least one said indication.

It is another object of the present invention to disclose the method, additionally comprising steps of providing said electrodes as either commercially available electrodes or proprietary electrodes

It is another object of the present invention to disclose the method, additionally comprising steps of determining the size of said patient's thoracic region by measuring at least one selected from a group consisting of: its width, its depth (front-to-back), its circumference, its perimeter length, its diameter, its radius, the length of an axis, its cross-sectional area, its surface area, its volume, and any combination thereof and any combination thereof.

It is another object of the present invention to disclose the method, additionally comprising steps of providing at least one mechanism for measuring the size of at least a portion of said thoracic region.

It is another object of the present invention to disclose the method, additionally comprising steps of selecting said mechanism for measuring the size of at least a portion of said chest from a group consisting of an anthropometer, a measuring tape and any combination thereof.

It is another object of the present invention to disclose the method, additionally comprising steps of providing at least one mechanism for reproducibly specifying at least one position on said thoracic region.

It is another object of the present invention to disclose the method, additionally comprising steps of providing said mechanism for reproducibly specifying at least one position on said thoracic region as a placement accessory adapted to ergonomically guide correct positioning of said electrodes and thereby enabled to consistently and repeatably place said electrodes at a predetermined position on the patient's thoracic region.

It is another object of the present invention to disclose the method, additionally comprising steps of providing an L-shaped placement accessory.

It is another object of the present invention to disclose the method, additionally comprising steps of providing a disposable placement accessory.

It is another object of the present invention to disclose the method, additionally comprising steps of providing said placement accessory comprising a slit of predetermined shape, thereby enabling marking of the surface of said patient's thoracic region.

It is another object of the present invention to disclose the method, additionally comprising steps of providing at least one mechanism for marking on the surface of said thoracic region in at least one predetermined position.

It is another object of the present invention to disclose the method, additionally comprising steps of selecting said mechanism for marking on the surface of said thoracic region from a group consisting of: a pen, a pencil, a marking pen, an IR laser marker, a temporary tattoo, a sticker, a frangible ink cartridge and any combination thereof.

It is another object of the present invention to disclose the method, additionally comprising steps of mounting at least one of said temporary tattoo, said sticker and said frangible ink cartridge on said placement accessory.

It is another object of the present invention to disclose the method, additionally comprising steps of comprising said placement accessory as part of said mechanism for marking on the surface of said thoracic region.

It is another object of the present invention to disclose the method, additionally comprising steps of providing a power supply adapted to power said screen, said controller, and to provide said current to said electrodes.

It is another object of the present invention to disclose the method, additionally comprising steps of providing, as a unit, a first kit comprising said measurement system.

It is another object of the present invention to disclose the method, additionally comprising steps of providing, as a unit, a second kit comprising at least one of a group consisting of: said mechanism for measuring the size of at least a portion of said chest, said mechanism for reproducibly specifying at least one position on said chest, and said mechanism for marking on the surface of said chest each said at least one position.

It is another object of the present invention to disclose the method, additionally comprising steps of determining said biological parameter from a calibrated voltage difference.

It is another object of the present invention to disclose the method, additionally comprising steps of selecting said calibration from a group consisting of: calibration to the size of the patient, calibration to the patient's breathing cycle, and any combination thereof.

It is another object of the present invention to disclose the method, additionally comprising steps of selecting said size calibration to be a linear calibration.

It is another object of the present invention to disclose the method, additionally comprising steps of calculating said size calibration from V_{c}=Vₘ−a(Pₘ−P_{c}), where a is a predetermined constant, Pₘ is the measured patient size and P_{c} is a predetermined standard cross-section size.

It is another object of the present invention to disclose the method, additionally comprising steps of selecting said measured patient size from a group consisting of: width of a predetermined portion of the body, circumference of a predetermined portion of the body, area of a predetermined portion of the body, thickness of a predetermined portion of the body, and any combination thereof.

It is another object of the present invention to disclose the method, additionally comprising steps of selecting said portion of the body from a group consisting of: a predetermined portion of the thorax, a predetermined portion of the height, a predetermined portion of the abdomen, and any combination thereof.

It is another object of the present invention to disclose the method, additionally comprising steps of selecting said calibration to the patient's breathing cycle from a group consisting of: calibration to a single breathing cycle, calibration to a predetermined portion of a breathing cycle, calibration to a plurality of breathing cycles and any combination thereof.

It is another object of the present invention to disclose the method, additionally comprising steps of calibrating to a single breathing cycle by averaging a plurality of voltage difference measurements, said voltage difference measurements taken during a single breathing cycle.

It is another object of the present invention to disclose the method, additionally comprising steps of selecting said average of said voltage difference measurements for a single breathing cycle from a group consisting of: the mean of the voltage difference measurements, the median of the voltage difference measurements and the mode of the voltage difference measurements.

It is another object of the present invention to disclose the method, additionally comprising steps of selecting said predetermined portion of a breathing cycle from a group consisting of: at least a portion of an inspiration, at least a portion of an exhalation, the beginning of an inspiration, the beginning of an exhalation, the end of an inspiration, the end of an exhalation, and any combination thereof.

It is another object of the present invention to disclose the method, additionally comprising steps of calibrating to said predetermined portion of a breathing cycle by averaging a plurality of voltage difference measurements, said voltage difference measurements taken either during a single breathing cycle or during a plurality of breathing cycles.

It is another object of the present invention to disclose the method, wherein said average of said voltage difference measurements for said predetermined portion of a breathing cycle is selected from a group consisting of: the mean of the voltage difference measurements, the median of the voltage difference measurements and the mode of the voltage difference measurements.

It is another object of the present invention to disclose the method, additionally comprising steps of calibrating to a plurality of breathing cycles by averaging a plurality of voltage difference measurements, each said voltage difference measurement being the minimum voltage difference measured during a single breathing cycle.

It is another object of the present invention to disclose the method, additionally comprising steps of selecting said average of said voltage difference measurements for a plurality of breathing cycles from a group consisting of: the mean of the voltage difference measurements, the median of the voltage difference measurements and the mode of the voltage difference measurements.

It is another object of the present invention to disclose the system, wherein said display unit (200) additionally comprises a means of holding at least one of said electrode leads (120) and said electrodes (110).

It is another object of the present invention to disclose the system, wherein said holding means comprises a member of a group consisting of: a recess in said display unit (200), a slot through said display unit (200), a clip, an elastic band, a strap, a buckle, and any combination thereof.

It is another object of the present invention to disclose the method, additionally comprising steps of providing a means of holding at least one of said electrode leads (120) and said electrodes (110).

It is another object of the present invention to disclose the method, additionally comprising steps of comprising said holding means of a member of a group consisting of: a recess in said display unit (200), a slot through said display unit (200), a clip, an elastic band, a strap, a buckle, and any combination thereof.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 schematically illustrates the reference planes used herein;

FIG. 2 depicts the components comprising the present system;

FIG. 3 depicts exemplary anthropometers;

FIG. 4 depicts exemplary methods of supporting the monitor unit;

FIG. 5 schematically illustrates a block diagram of the monitor unit;

FIG. 6 schematically illustrates a block diagram of the processing unit;

FIG. 7 schematically illustrates a block diagram for the control software for the system;

FIG. 8 depicts an embodiment of the Start New Session screen;

FIG. 9 depicts an embodiment of one of the setup screens, the Quick Guide screen;

FIG. 10A-C depicts an embodiment of the System Setup screen;

FIG. 11 depicts an embodiment of the screen during monitoring;

FIG. 12 schematically illustrates an embodiment of an adjustable placement accessory;

FIG. 13 schematically illustrates a method of marking the placement of the paired electrodes on the patient's skin;

FIG. 14 schematically illustrates a user placing the paired electrodes in position on a patient;

FIG. 15 schematically illustrates a method of locating the correct position in which to place the unpaired electrode;

FIG. 16 schematically illustrates an embodiment of an electrode pair;

FIG. 17 schematically illustrates properly and improperly placed electrodes; and

FIG. 18 schematically illustrates proper placement of the anthropometer.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for system and method for monitoring and evaluating biophysical measurements in the body. In particular, the disclosure relates to a system where electrodes are placed on the skin in a predetermined pattern to enable measurement of biophysical parameters of the thorax.

The term ‘electrical impedance tomography’ or ‘EIT’ hereinafter refers to a medical imaging technique in which an image of the conductivity or permittivity of part of the body is inferred from surface electrical measurements. Typically, conducting electrodes are attached to the skin of the patient and small alternating currents are applied to some or all of the electrodes. The resulting electrical potentials are measured, and the process may be repeated for numerous different configurations of applied current. From the measured potentials, the inverse problem of finding the resistivity of every pixel/voxel as a function of position within the body from the applied currents and measured potentials is solved.

The term ‘parametric electrical impedance tomography’ or ‘pEIT’ hereinafter refers to an EIT technique wherein, as a simplifying approximation, each biophysical unit (a tissue, an organ, or a component within the organ) in, for example, the thorax, will be assigned a single resistivity value for the entire unit.

From the measured potentials and from knowledge of the geometry (sizes, shapes and locations of organs), the inverse problem of finding the resistivity parameters of every bio-physical unit as a function of position within the body from the applied currents and measured potentials is solved.

This predefined parameterization improves the robustness of the solution compared to EIT and reduces its sensitivity to noise and proneness to ill-posed solutions.

In the case of the thorax, in the simplest pEIT system, the thorax would consist of two parameters—the left lung resistivity and the right lung resistivity, while resistivity values of all other tissues are pre-assigned. In this example, pEIT would solve the inverse problem and, using two independent potential measurements, would compute the two resistivity values, one for each lung.

The term ‘fluid status’ hereinafter refers to a measure adapted to compare the volume of fluid in the lung with a predetermined expected volume of fluid, typically at times when no treatment intended to change the lung fluid status is being used on the patient. In a normal human, the predetermined expected volume of fluid would be zero or a nominal value.

The term ‘fluid volume’ hereinafter refers to the volume of fluid in at least a portion of the lung.

The terms “fluid volume” and “lung fluid volume” will hereinafter be used interchangeably.

The term ‘resistance’, R, is the resistance to the flow of current. The inverse of resistance is conductance, S; S=1/R; and resistance and conductance will be used herein as equivalents.

The term ‘breathing cycle’ hereinafter refers to a single inhalation/exhalation event.

The term ‘resistivity’, ρ, is the intrinsic property of the material, the resistance to the flow of current across a unit area per unit length. The inverse of resistivity is conductivity, σ; σ=1/ρ; and resistivity and conductivity will be used herein as equivalents.

The term ‘closed loop’ hereinafter refers to a control system with an active feedback loop, that automatically changes the input based on the difference between a current input signal and a feedback signal dependent on the difference between the actual output and a desired output. For non-limiting example, in a closed-loop system of treating a patient, the treatment is applied to the patient and at least one criterion of the patient is measured, the criterion being something that is affected by the treatment. (In the present system, the criterion is the volume of fluid in the lungs.) The criterion is then compared to an expected value and the difference between the measured and expected values is used to determine whether the treatment should be changed and, if so, how it is to be changed and by how much it should be changed. The system then applies the modified treatment to the patient.

The term ‘open loop’ hereinafter refers to a control system without an active feedback loop. For non-limiting example, in an open control system of treating a patient, the treatment is applied to the patient and at least one criterion of the patient is measured, the criterion being something that is affected by the treatment. The measured criterion can be displayed for the use of a physician or caregiver, or it can be stored for later reference. It can also be used to determine recommended changes to the treatment, which can be displayed for the use of a physician or caregiver. However, since the system does not apply the changed treatment, the control loop is open since the changes to the treatment that actually occur are made by the physician or caregiver, independent of the system.

The term ‘bio-impedance’ hereinafter refers to the response of a living organism (or a portion thereof, such as a body part, organ, tissue, or the like) to an externally applied electric current. It is a measure of the opposition to the flow of that electric current through the tissues. The measurement of the bio-impedance (or bioelectrical impedance) has proved useful as a non-invasive method for measuring various parameters of the body.

The term ‘electrocardiographic body surface mapping’ or ‘BSM’ hereinafter refers to an electrocardiographic (ECG) technique that uses multiple (generally 80 or more) electrocardiography leads to detect cardiac electrical activity. Body surface potential maps (BSMs) depict the time varying distribution of cardiac potentials on the entire surface of the torso. BSM's in general, contain more diagnostic information and can provide improved diagnostic accuracy compared to the standard ECG.

The term ‘thoracic region’ hereinafter refers to the region between the abdomen and neck wherein the ribs are located.

The term ‘anthropometer’ hereinafter refers to an instrument used for measuring the human trunk and limbs. It typically consists a calibrated rod to which are attached two arms, one fixed and one movable along the calibrated rod. Anthropometers function in a manner similar to caliper devices used for lab mechanical measurements.

The present invention provides a system and method of measuring biophysical parameters of the human body, preferably of the thoracic region, using at least one pair of electrodes, preferably placed on the skin, to inject current into the body and at least one second pair of electrodes, also preferably placed on the skin, to measure the potential difference generated in the body between the second pair of electrodes by the current injected by the first pair.

In preferred embodiments, a biophysical parameter to be measured is the pulmonary edema, the volume of extracellular fluid in the lung.

In some embodiments of the system, the EIT technique or the pEIT technique is used to determine the volume of extracellular fluid. The fluid volume or a parameter related to it such as resistivity is monitored; treatment can be instituted or changed based on the physician's or other carer's assessment of the situation. In some embodiments, in addition to monitoring the fluid volume, the system provides an open-loop assessment of the effect of treatment administered to the patient, generating and displaying an assessment of the effectiveness of the treatment, based on a comparison of the patient's response to the responses of previous patients to the same treatment. In other embodiments, the system provides a closed-loop assessment of the effect of treatment administered to the patient, based on a comparison of the patient's response to the responses of previous patients to the same treatment. In the closed-loop embodiments, the system titrates drug to the patient, based on the assessment of the effect of treatment. In such closed-loop embodiments, for non-limiting example, the system can reduce the dosage of the drug if the patient responds to the treatment more rapidly than the norm of the previous patients, or it can increase dosage of the drug if the patient is responding more slowly than the norm.

In the EIT technique, the region of the body of interest is treated as a space filled with materials of variable resistivity. The size and shape of the space is provided by another means, for non-limiting example, X-ray CT images. No assumptions are made as to the resistivity values at any point within the space. The space is subdivided into voxels (volume cells), with each voxel having a known position and known dimensions, with the voxels filling the space. It is assumed that each voxel is small enough that the resistivity within the voxel is substantially constant, so that it is reasonable to assign a single resistivity parameter to each voxel. These resistivity parameters are unknown and must be solved for.

In order to find the resistivity parameters, a number of measurements are made of potential difference resulting from applied currents, as described in more detail hereinbelow. From these measured potentials, the inverse problem is solved of finding the resistivity parameters for every voxel from the applied currents and measured potentials.

In the full inverse problem, a set of equations are built that connect the voxel resistivity values and the expected potential developed at the given points, the points where the electrodes are located. In these equations, every equation represents an independent measurement, where an independent measurement is a specified combination of current injection using one pair of electrodes and potential measurement using another pair of electrodes. In order to properly solve the set of equations, one independent measurement is needed for each voxel. Therefore, if the space is subdivided into N voxels, N independent measurements are needed so that, the smaller the voxel size, the larger the number of independent measurements required. For each measurement, a pair of electrodes (the “active pair”) is used to apply AC current and a different pair of electrodes (the “passive pair”) is used for measuring the potential developed on the surface. Therefore, the maximum number of independent measurements possible with a given set of electrodes will depend on the number of ways in which it is possible to pair up pairs of electrodes. The theoretical maximum number of ways, Mₘₐₓ of creating two pairs of electrodes from a number n of electrodes is

$$\begin{matrix}{M}_{max} = ( \begin{matrix}n \\ 2\end{matrix} ) ( \begin{matrix}n - 2 \\ 2\end{matrix} ) \\ = ( \frac{n !}{2 ! ( n - 2 ! )} ) ( \frac{( n - 2 ) !}{2 ! ( n - 4 ! )} ) \\ = \frac{n ( n - 1 ) ( n - 2 ) ( n - 3 )}{4}\end{matrix}$$

However, in practice this theoretical maximum can not be reached, since some of the measurements are not independent. For example, in a system with 5 electrodes, 3 of the possible sets of pairings are: set 1: {1,2} and {3,4}, set 2: {1,2} and {3,5} and set 3: {1,2} and {3,5}. However, these three sets of pairs of electrodes only provide two independent measurements, since the equation for set 3: {1,2} and {3,5}, can be derived from the equations for sets 1 and 2.

Depending on the exact setup used, in some embodiments, the practical maximum number of independent measurements M_{max,p,i} is

$${M}_{max , p , 1} = \frac{n ( n - 3 )}{2}$$

where n is the number of electrodes.

In preferred embodiments, the practical maximum number of independent measurements M_{max,p,2} is

M_{max,p,1}=(n−1)(n−3)

where n is the number of electrodes.

If the maximum number of independent measurements available with a given set of electrodes is insufficient for the desired resolution, more electrodes are needed. For example, for a typical adult male thorax divided into relatively large 5 cm×5 cm×5 cm voxels, on the order of 200 measurements would be needed and, therefore, in preferred embodiments, more than 16 electrodes would be needed. In other embodiments, twice this many, more than 32, electrodes would be needed. In either case, applying this large number of electrodes would be trying both to the user applying them and to the patient to whom they are being applied. Since the electrodes must be applied accurately in order to get accurate results, the process becomes impracticable.

In addition, as is well-known, inverse problems with large numbers of unknown parameters are ill-posed; they are difficult to solve and are very sensitive to noise and measurement error—small measurement errors or small changes in a reading due to noise can have a large effect on the results.

In the pEIT technique, as a simplifying approximation, a single value of resistivity is assigned to each organ in the region of interest, for example, the thorax. The resistivity values of all organs except for the organs of interest are predefined. For example, for the thorax, the resistivity values of all organs therein are predefined, except for the resistivity values of the lungs, which are variable parameters.

In order to solve the system of equations, the same number of equations and free parameters is needed. However, since in pEIT the number of free parameters has been very much reduced because many of the resistivity values are predefined, the number of equations has been much reduces, so that the number of measurements and the number of electrodes that are needed is also greatly reduced improving computation cost and stability, and avoiding the tendency to ill-posed solutions.

From the measured potentials and from knowledge of the geometry (sizes, shapes and locations of organs), the inverse problem of finding the resistivity parameters of every bio-physical unit as a function of position within the body from the applied currents and measured potentials is solved.

This predefined parameterization improves the robustness of the solution compared to EIT and reduces its sensitivity to noise and proneness to ill-posed solutions.

A pEIT system uses a pre-defined model of the geometry, identifying where the geometry is (3D contours of lungs, heart, etc.). The geometry can be defined with more or less sophistication. For example: In the least sophisticated model, the two lungs are treated as a single computational unit with a single (average) resistivity value. In a typical model, each lung is a computational unit and each has a single resistivity. In a more sophisticated model, the lungs are split into lobes and the pleura are identified, resulting in having a plurality of resistivity values for each lung.

pEIT can consider geometrical changes over time, such as the end-systole and end-diastole phases of the heart, or it can be simplified and consider the geometry in an average state. Similarly, for the lungs, an average geometry can be used or separate geometries can be used for the expanded lungs at the end of an inhalation and the contracted lungs at the end of an exhalation. Another example of non-fixed geometry: pEIT can account for the enlargement of the heart, as part of the acute HF pathology. A sophisticated model can account for this, by assuming that the heart's size changes as a function of the lungs' resistivity.

As well as geometry changes, pEIT can consider changes in the relative amounts of air and fluid in the lungs. The geometrical units comprising the lungs, as described above, can have a single resistivity, whatever the stage of the breathing cycle, or different resistivities can be used for the expanded lungs and the contracted lungs, with the difference in resistivity between the expanded lung and the contracted lung being attributed at least partly to the known difference in resistivity between lung fluid and air.

In pEIT, each bio-physical unit (organ or sub-organ) is assigned a resistivity value. For some, the resistivity is a fixed value, typically, a known value found from the literature. For others, the value is variable, a parameter to be determined by the pEIT process.

To summarize, pEIT can be implemented at several levels of sophistication. For the thorax, these are listed below in order from the simplest to the more sophisticated:
1. The simplest pEIT system finds only one parameter—the average resistivity of the lungs, while assuming values for all other organs, and the geometry of all organs including the lungs. This requires only a single equation and therefore one measurement using only 4 electrodes.
2. A more sophisticated system computes resistivity independently for each lung. This requires two equations (one for each lung) and, therefore, two independent measurements. It therefore requires a minimum of four electrodes.
3. In preferred embodiments, the present system uses three independent measurements, and five electrodes, in order to achieve better reliability with unilateral conditions.
4. Future embodiments can include more lung regions: using five or more electrodes, more regions can be identified, for example the lung lobes or the pleura.
5. Another embodiment can include cardiac output: an eight electrode model can provide left lung resistivity, right lung resistivity, 3 axes of the heart in end systole and 3 axes in end-diastole.

In all EIT and pEIT systems, it is almost impossible to get accurate measurement of the lung fluid without accurate knowledge of the locations of the electrodes with respect to the body. In the present system, a patient measurement system is used to ensure that the electrodes are accurately positioned.

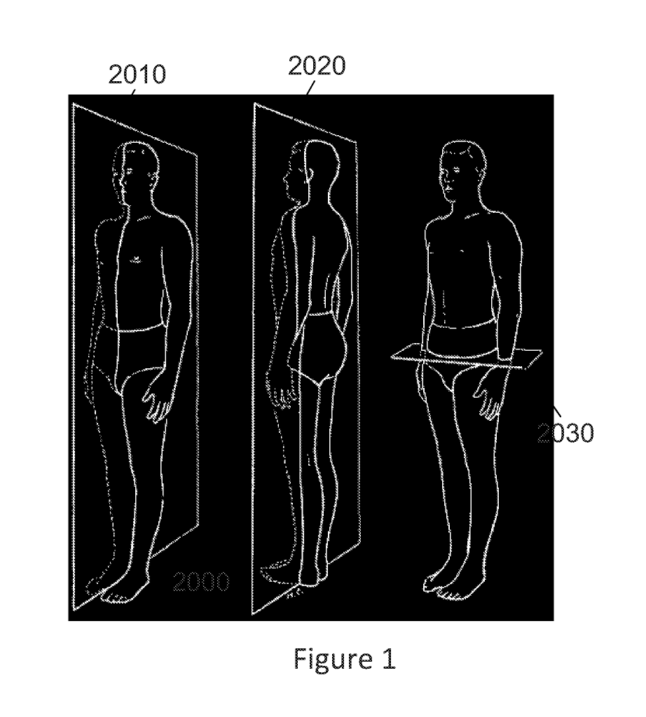

In reference to FIG. 1, an object (2000) (a human in the figure) has three perpendicular cross-sections. Herein, these three perpendicular sections will be referred to as the median plane (2010, FIG. 1A), the frontal plane (2020, FIG. 1B), and the transverse plane (2030, FIG. 1C). The median plane (2010) is a longitudinal plane splitting the object into two approximately antisymmetric halves. As used hereinbelow. ‘sagittal plane’ will be refer to any plane parallel to the median plane. The coronal plane (2020) divides the front of the object from the back of the object and is a longitudinal plane perpendicular to the median plane (2010). The transverse plane (2030) is a plane dividing the distal end (in FIG. 1C, the head end) of the object from the proximal end (in FIG. 1C, the foot end) of the object, and is a plane perpendicular to both the median plane (2010) and the coronal plane (2020).

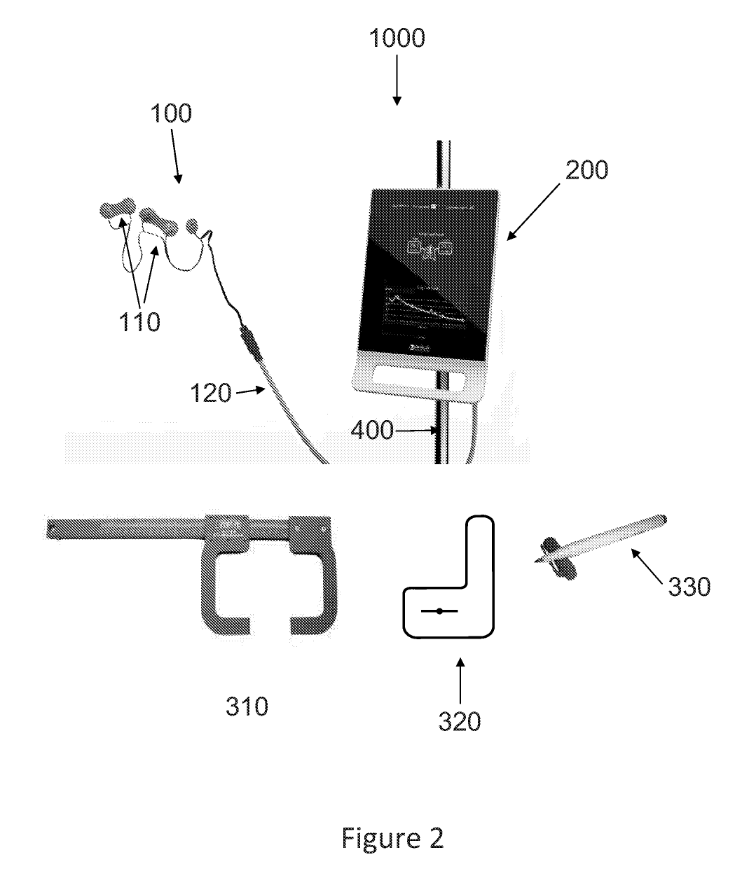

FIG. 2 shows an embodiment of the system (1000) of the present invention. It comprises three main parts, a monitor unit (200), a set of accessories (100) comprising a set of electrodes (110) and associated leads (120), and a patient measurement system to determine where to place the electrodes.

In the embodiment shown in FIG. 2, the monitor unit (200) comprises a power cord (271) connected to an electric power supply, a data bus (291), preferably a USB data bus, to transfer data, and a holder (225) in which the electrode (120) connector can be safely stored until connection, thus eliminating the possibility of misplacing the electrodes during the early phases of setup.

In the embodiment shown, the holder comprises a slot through the perimeter of the tablet PC. In other embodiments, the holder can be selected from a group consisting of: a recess in said display unit (200), a clip, an elastic band, a strap, a buckle, and any combination thereof, alone or in combination with the slot.

In the embodiment shown in FIG. 2 (1000), the patient measurement system comprises three parts: an anthropometer (310) to measure the size of the thorax; a placement accessory (320); and a skin marker (330) to mark the patient's skin in the positions indicated by the placement accessory (320), thereby ensuring that the electrodes are accurately placed on the skin, ensuring an accurate measurement of the patient's lung fluid.

In preferred embodiments, a fixed size placement accessory is used, so that the electrodes are placed a predetermined, fixed distance below the armpit and there is a predetermined, fixed distance between the electrodes. The patient size, as determined by the anthropometer, is then used by the software to normalize the results for the patient's size.

In other embodiments, the placement accessory is sizeable to the patient based on the patient's size, as determined by the anthropometer (310), so that normalization to the patient's size is provided by the placement accessory.

In preferred embodiments, the placement accessory (320) is disposable.

In preferred embodiments, an anthropometer is used to measure the size of the thorax. In other embodiments, a measuring tape or other means of measurement as is known in the art is used.

In some embodiments of the system, the electrode set (110) and the leads (120) are commercial, off-the-shelf items. In preferred embodiments, custom electrodes are used. In preferred embodiments of the system, the electrode set is prewired with five electrodes, the wiring having a single connector and the electrodes comprising a long-term (48-72 hour) hydrogel adhesive. The electrodes are labelled to indicate their intended position on the body. In some embodiments, the lead is 2.5 m long, although any other length can be used.

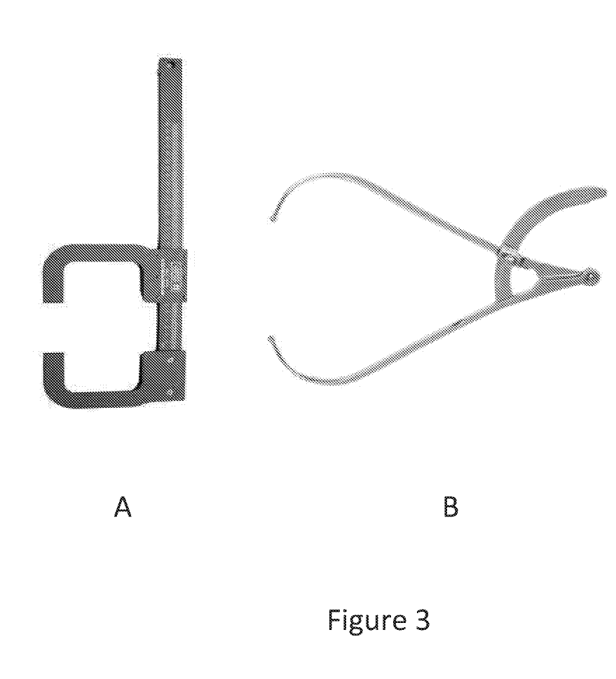

The anthropometer is preferably an off-the-shelf unit, preferably intended for medical use. Exemplary anthropometers are shown in FIG. 3; any type of anthropometer known in the art that can accurately and reliably measure objects of the size of the human thorax can be used.

The monitor unit (200) comprises a display unit comprising a screen and preferably incorporating a processing unit, preferably a tablet PC, a power supply, and the software, firmware and hardware needed to implement the system. In preferred embodiments, the software is loaded into the processing unit and associated firmware and hardware is embodied in a PCB which attachable to or embedded in the display unit, such as a tablet PC. In other embodiments, the software remains in the PCB while the processing occurs within the processing unit in the display unit. In yet other embodiments, the PCB comprises a processing unit; most processing occurs in the PCB and the display unit is used primarily for graphics processing and display.

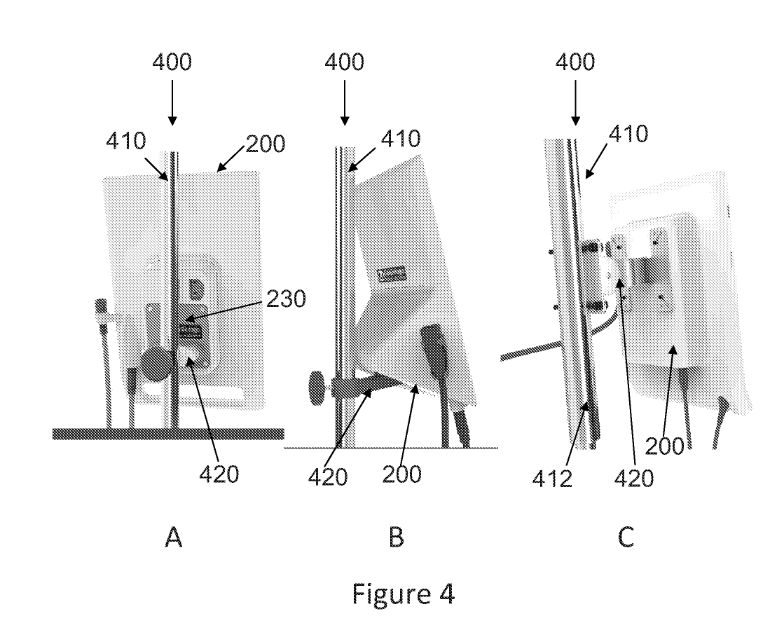

In reference to FIG. 4, in preferred embodiments, a support system (400) for the monitor unit (200) is provided. In preferred embodiments, the support system (400) comprises a vertical member and a support mechanism.

In the embodiments shown in FIG. 4A-C shows exemplary support systems (400). All three support systems (400) comprise a conventional vertical member (410), and a conventional floor-based support unit (not shown) such as but not limited to a tripod or horizontal support plate.

In the embodiment shown in FIG. 4A, a support plate (230) is attached to the back of a standard tablet PC (200) and is attachable to a clamp (420), with the support plate (230), adapted to hold the tablet PC firmly attached to the clamp. The clamp (420) is attachable to the support member (410), and holds the PC (200) in a fixed position for use.

In the embodiment shown in FIG. 4B, the monitor unit (200) is a purpose-built unit, with the clamp mechanism (420) an integral part of the monitor unit (200).

In the embodiment shown in FIG. 4C, a support plate (230) is attached to the back of a standard tablet PC (200) and is attachable to a clamp (420), with the support plate (230), adapted to hold the tablet PC firmly attached to the clamp. The clamp (420) is attachable to the support member (410), and holds the PC (200) in a fixed position for use. In the embodiment shown in FIG. 4C, the clamping unit clamps to guides (412) on the sides of the monitor unit (200), thereby stabilizing the monitor unit and making it easier to raise and lower the monitor unit (200).

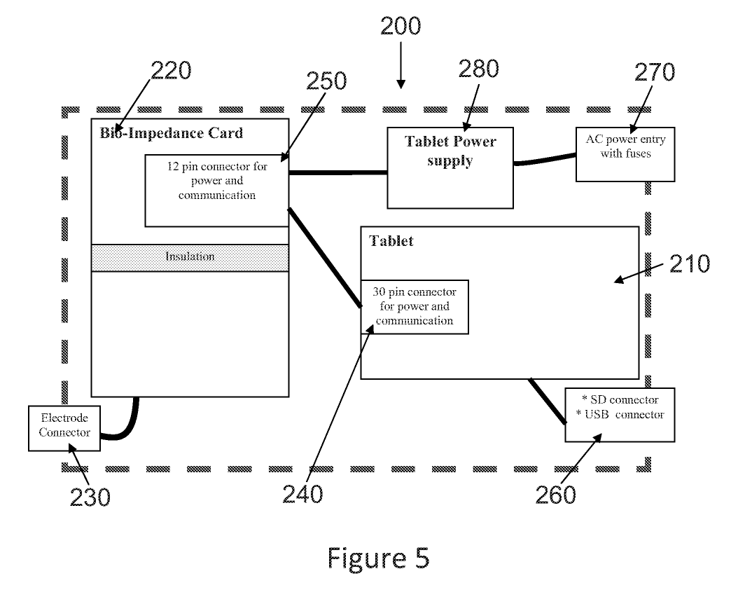

FIG. 5 shows a block diagram of the tablet PC and associated electronics (200) for the system. The monitor unit comprises a display unit (210), preferably a tablet PC. The display unit (210) is connected to the bio-impedance card (220) providing software, firmware and hardware for the bioimpedance sampling via two connectors. The first connector (240) is on the tablet PC (210), and is, in this embodiment, a 30 pin connector. The second connector (250) is on the bio-impedance card (220) and is, in this embodiment, a 12 pin connector. A further connector (260) provides SD and USB connections. On the bio-impedance card (220) is an external connector (230), to enable the electrodes (110, not shown) to be connected to the system (200).

In further reference to FIG. 5, power for the system is provided by a standard AC power supply (270). Power from the standard AC power supply (270) is passed through a transformer/regulator, the tablet power supply (280), and from thence, via, in this embodiment, the 12-pin connector (230) powers the system (200).

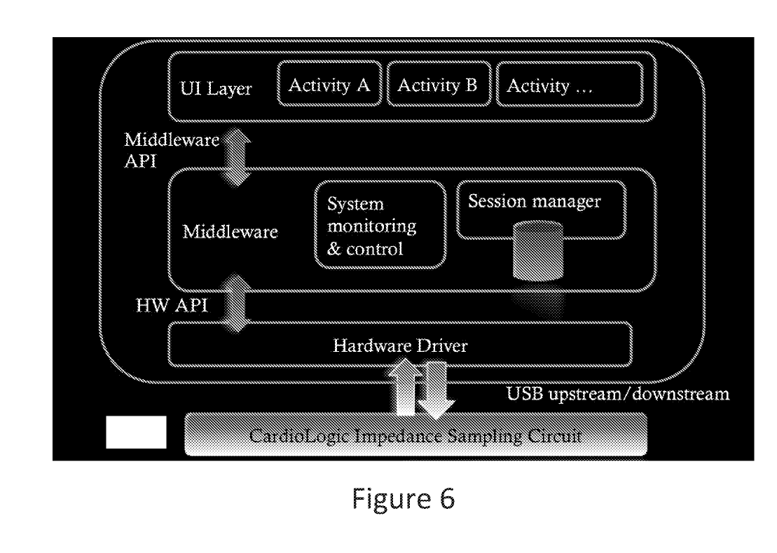

A block diagram of the processing unit layers is shown in FIG. 6. The uppermost layer (UI layer) comprises the I/O, wherein current activities (Activity A, Activity B, etc.) are displayed and commands for possible future activities (Future Activity) are accepted.

The UI layer:
Displays data, graphs and visualization
Receives commands from the user (for example “start a monitoring sessions”), and data entered by the user
Creates reports and prints or saves them to external media, and
Communicates with the middleware for fetching data from the database or storing data in the database.

Supporting the uppermost UI layer is the middleware API comprising the middleware—the system monitoring and control software and the session management software, which manage the data in the system.

The Middleware layer:
Receives the data stream from the sampling circuit as it is being measured,
Does algorithm computations and saves the outputs, parameters such as resistivity, conductivity, respiratory rate, gradients and integrals, in the session database, and
Retrieves current or historical data for applications in the UI layer on demand; for example: the most recent 5 values of respiratory rate, the vector of values of left-lung fluid index measured between July 5 at 4:24 AM and 7:37 AM.

The middleware layer communicates with the hardware API layer, which is responsible for communication between the sampling hardware and the PC. The hardware API layer comprises the hardware drivers, and controls the CardioLogic impedance sampling circuit.

The Hardware API:
Monitors the status of the monitoring session (idle, in progress, faulty) and the status of self tests done by the sampling hardware
Monitors the status of the electrodes, and verifies their connection integrity, and
Receives the data as a stream as it is being measured/created by the sampling hardware

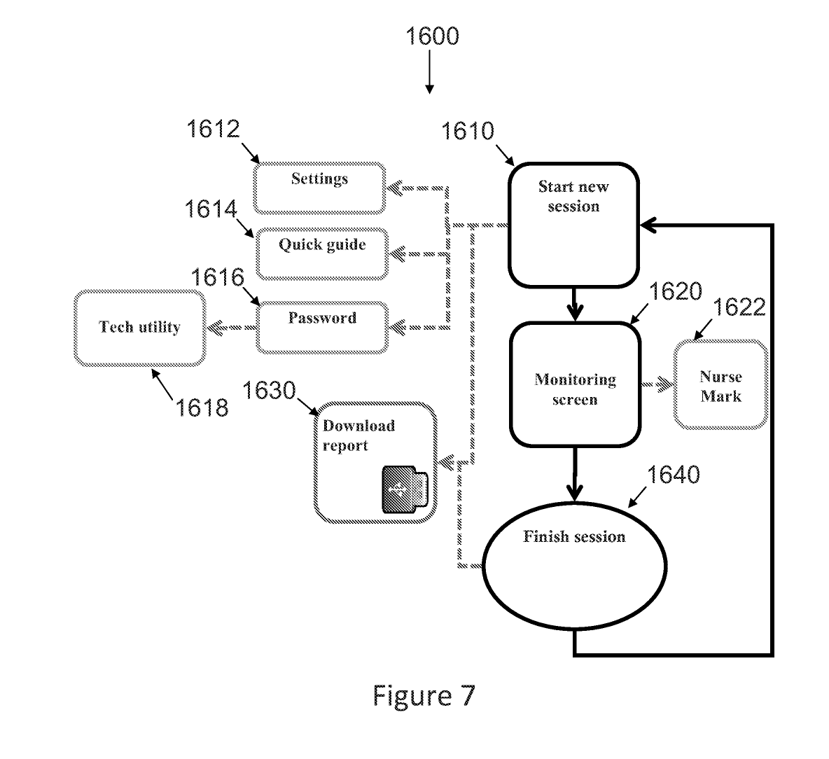

FIG. 7 shows an embodiment of a block diagram (1600) for the control software for the system. When the system is turned on, a screen appears which enables the user to start a new session (1610). The user can display a guide (1614) for using the system, provide settings (1612) for various parameters for the session, as described hereinbelow, or enter a password (1616) and then set advanced features of the system. At this time, the user can also identify a previous session and download a report (1630) on the previous session. After setting up the controls for the current monitoring session and attaching the electrodes to the patient's body, the user instructs the system to start monitoring. The system starts measuring the patient's biophysical parameters, as instructed, and displays a monitoring screen (1620), as described hereinbelow. At any time during a monitoring session, a user can place a “nurse mark” (1622), where the user enters a comment into the system. In preferred embodiments, an identifying mark will appear on the display, indicating that a comment has been entered. Touching the nurse mark enables a user to read the comment.

At the end of a monitoring session, a further screen appears, enabling the user to finish the session (1640). From this screen (1640), the user can close down the system, start a new session (1610), or download a report (1630), either of the current session or of a previous session.

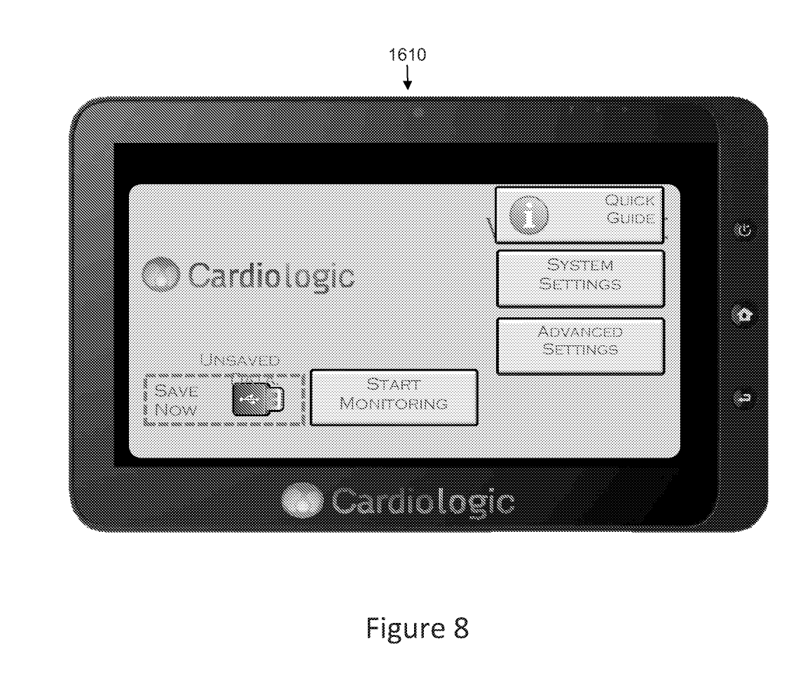

FIG. 8 shows an embodiment of the Start New Session screen (1610). The button “Quick Guide”, as described hereinabove, provides access to a quick guide to the use of the system. The button “System Settings” enables the user to set up a session, while the “Advanced Settings” button enables the user to alter password-protected settings. The screen further enables a user to save data, and to start a monitoring session.

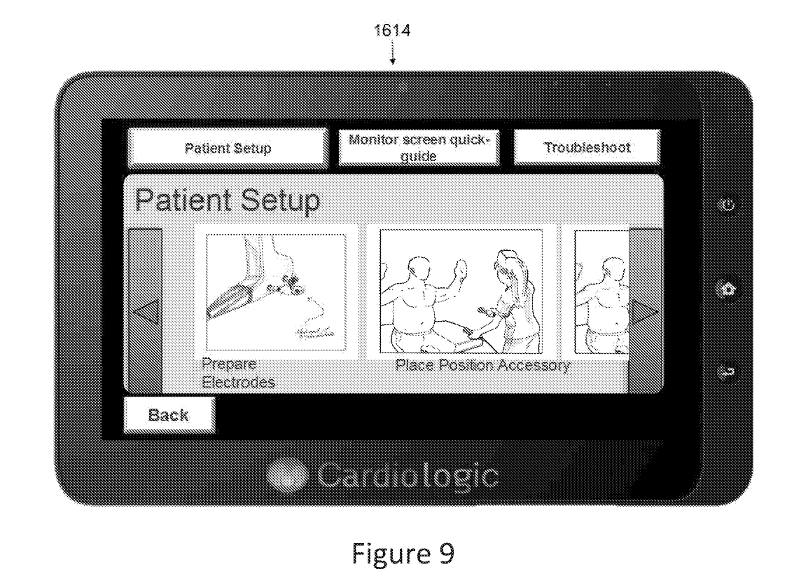

FIG. 9 shows an embodiment of the quick guide screen (1614). This provides three options. Patient Setup, Monitor Screen Quick Guide and Troubleshoot. Patient Setup, a part of which is displayed in FIG. 9, guides the user through the process of preparing the electrodes and placing them in the proper positions on the patient's body. Monitor Screen Quick Guide provides an overview of the capabilities of the system and the meanings of the icons on the screen. The Troubleshoot button provides troubleshooting advice and guides the user through identifying the causes of problems such as, for non-limiting example, lack of signal from the electrodes.

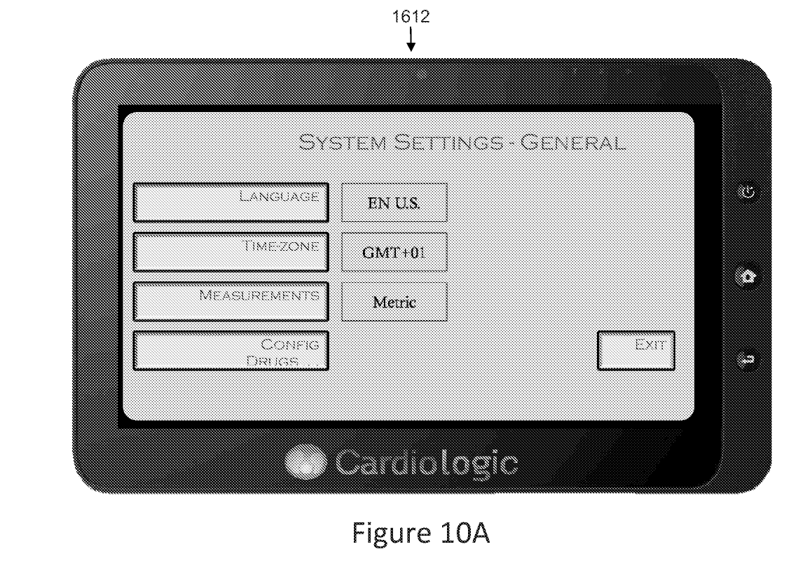

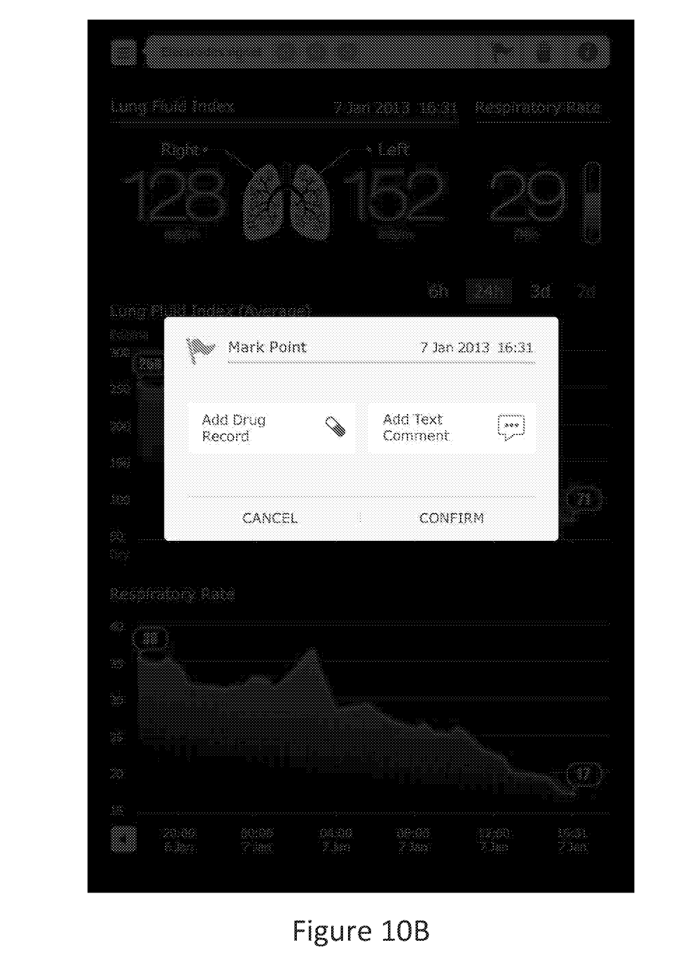

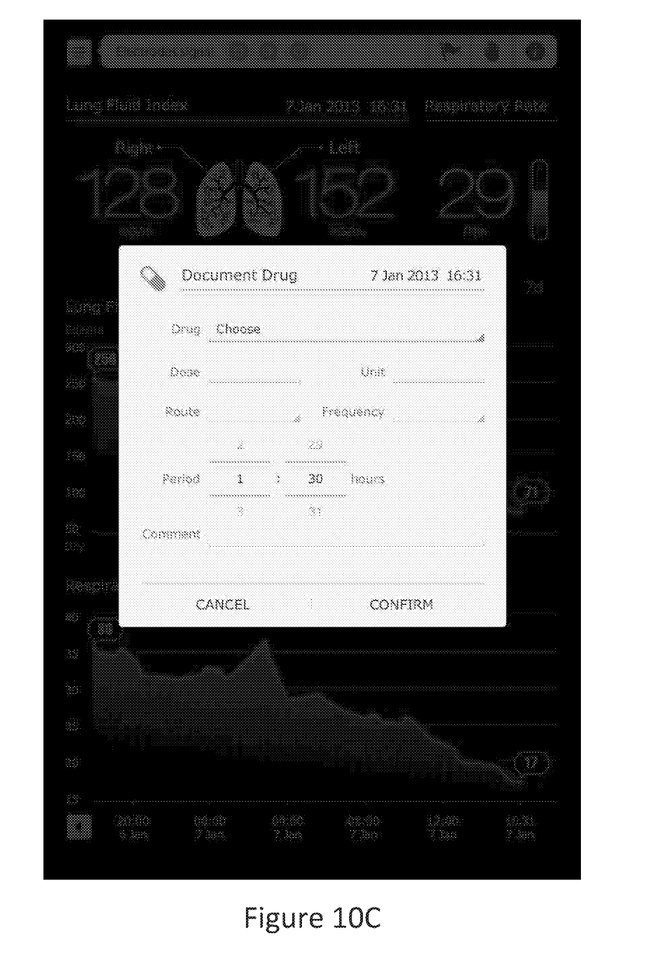

FIG. 10A-C shows an embodiment of two of the setup screens (1612). In FIG. 10A, the parameters to be set are: the language to be used (a typical example, U.S. English, is shown); the time zone in which the session occurs (example, GMT+01); the units system for the measurements (example, metric), and the drug(s) to be given to the patient and the schedule for administration of the drug(s). In this embodiment, pressing “Config Drugs” brings up another screen (1612, FIG. 10B) which enables the user to accept the predetermined drug regimen, or to alter it.

FIGS. 10B and 10C shows embodiments of screens which enable a user to specify the drug regimen for the patient during the current session. In monitoring-only embodiments of the system, the drug regimen is stored but not displayed. In open-loop embodiments, the drug regimen is displayed and, in some variants, the user is reminded to administer the drug(s). In closed loop embodiments, the system administers the drugs, based on the drug regimen, at the appropriate times.

FIG. 10B shows an embodiment of a screen that enables a user to enter a drug regimen or enter a comment. After the user chooses to enter a drug regimen, a screen such as the embodiment shown in FIG. 10C is displayed, which enables the user to enter the type of drug, preferably from a drop-down list, the dosage of the drug and the units of the dosage (for non-limiting example, a dose could be entered as 600 μg or as 0.6 mg). The user can also enter the route of administration, for non-limiting example, orally, parenterally, transdermally, or intravenously, the frequency of administration, the duration of the treatment (the period), and, if desired, a comment.

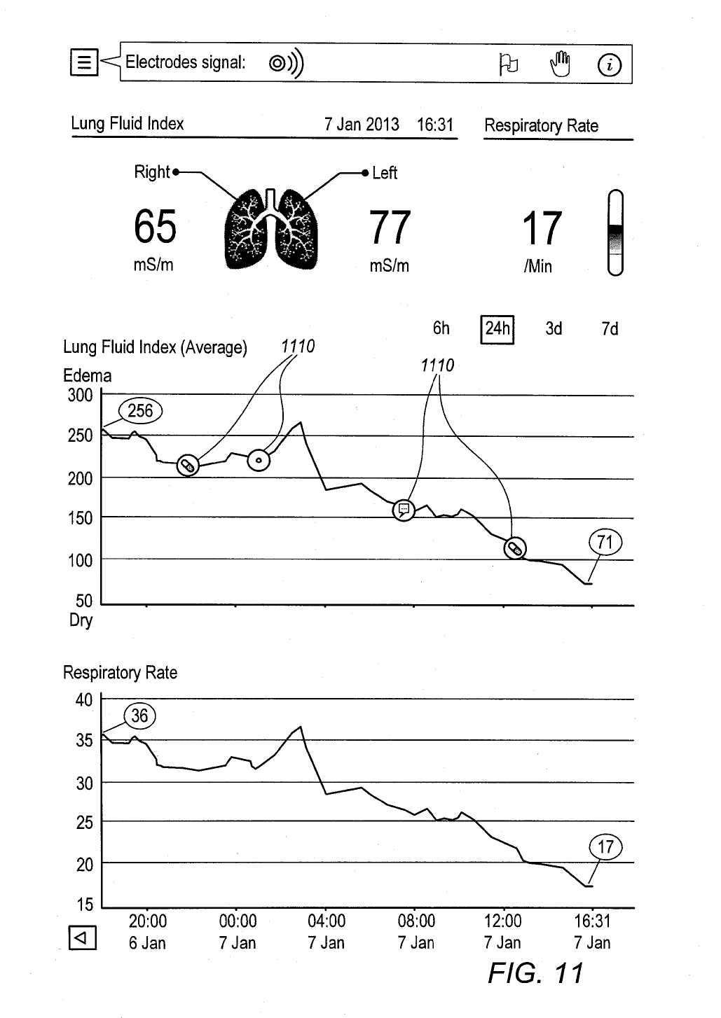

An “event”, a drug regimen, comment or time point, can be entered at any time during monitoring. Once it has been entered, a “nurse mark” is placed on the graph (see FIG. 11 hereinbelow) indicating the time the event was entered. Nurse marks can be a detailed report of medication, as described above, a comment, or merely a time point.

The user can access the event, preferably by touching the event marker on the screen. Access to the event can be, for non-limiting example, by displaying the event data in a new window or in a pop-up window.

Since nurse marks will identify the time(s) a drug is administered and the drug(s) administered, the user can quickly identify changes in the patient's condition due to the administration of the drug(s), making it easier to determine, for non-limiting exam=le, whether the patient's response to the drug(s) is normal or abnormal.

Other parameters that can be entered, via another screen or screens, include, but are not limited to, the patient's name, I.D. number, patient's identification number within the hospital, date of admission, name or other identifier for the hospital, name or other identifier for the user administering the session, patient's height, weight, thorax size (width, thickness, perimeter length), and patient's clinical signs at the start of the session.

FIG. 11 shows an embodiment of a monitoring system in progress. In this embodiment, at the center and bottom of the screen is shown a graph of the fluid content of the lungs as a function of time (“Lung Fluid Index (Average)”), and, at the bottom of the screen is shown the patient's respiratory rate as a function of time (“Respiratory Rate”). The measured values of the current fluid volumes of the two lungs and the current respiratory rate are also shown, at the top of the screen. In this embodiment, the fluid volume is indicated by the conductivity of the lungs (in mS/m), which is proportional to the fluid volume.

In preferred embodiments, the user can switch between the monitoring screen as shown and a similar screen in which a lung fluid volume graph is displayed, but not a respiratory rate graph. Other monitoring screens include, but are not limited to, a screen in which there is a separate lung fluid volume graph for each lung or portion thereof, or a screen in which several lung fluid volume curves are presented on the same graph. Other arrangements of the monitoring screen will be obvious to one skilled in the art.

In preferred embodiments, all graphs on the monitoring screen are displayed such that their X-axes are synchronized; for any vertical line drawn on the screen, points on different curves that lie on that vertical line occurred at the same time. In the exemplary monitoring session shown in FIG. 11, the most recent time is 16:31 on 7 January. At that time, the average lung fluid volume was 71 mS/m and the respiratory rate was 17/min.

In preferred embodiments, the default option is to display all of the data, at the largest scale compatible with displaying data for the entire time of monitoring. In preferred embodiments, four time scales are provided, 6 hr, 24 hr, 3 d and 7 d. After 6 hours of monitoring, the display switches automatically to the 24 hour scale. Similarly, after 24 hours of monitoring it switches to the 3 day scale and after 3 days, it switches to the 7 day scale. In other embodiments, other scales can be used as appropriate.

A user can switch manually between scales so that, for non-limiting example, the user can manually switch to the 6 hour scale during the fourth day of treatment. In cases such as this, where only a portion of the graph can appear on the screen, the user can scroll through the graph. Therefore, the user can, at any time, view any portion of the data on the largest scale provided. In the example above, the user can, for non-limiting example, scroll through until the hours from 04:00 to 010:00 on the third day of monitoring are displayed on the screen.

In preferred embodiments, after a predetermined time wherein the user has not affected the display, a predetermined “idle time”, the display reverts automatically to the default display. In some preferred embodiments, this predetermined time is 3 minutes.

In preferred embodiments, a second monitoring screen (not shown) displays ventilation visualizations.

A session report can be generated, displayed and saved. As is known in the art, means of storage include, but are not limited to, saving the report to disk or other storage medium as a stand-alone item, adding it electronically to the patient's file, transmitting it to other medical practitioners, or printing it.

In use, after starting a monitoring session, the user measures the size of the patient's thorax using anthropometer (310). The size can be defined by the width of the thoracic region, the depth (front-to-back) of the thoracic region, the circumference of the thoracic region, the area of the thoracic region, or any combination thereof. At least one placement accessory (320) is then sized to the patient's thoracic region, by adjusting the placement accessory (320) size, for example, by removal of parts of the placement accessory (320), or by selecting an appropriate set of markings on the placement accessory (320). In some embodiments, there are two placement accessories (320), one for the left side of the body and one for the right side.

The placement accessory is shaped such that it ergonomically guides correct positioning and enables consistent and repeatable placement of the electrodes (110) at the same position on the torso surface. Consistent and repeatable placement of the electrodes (110) can be ensured by the shape of the placement accessory, by markings on the surface of the placement accessory, and by any combination thereof.

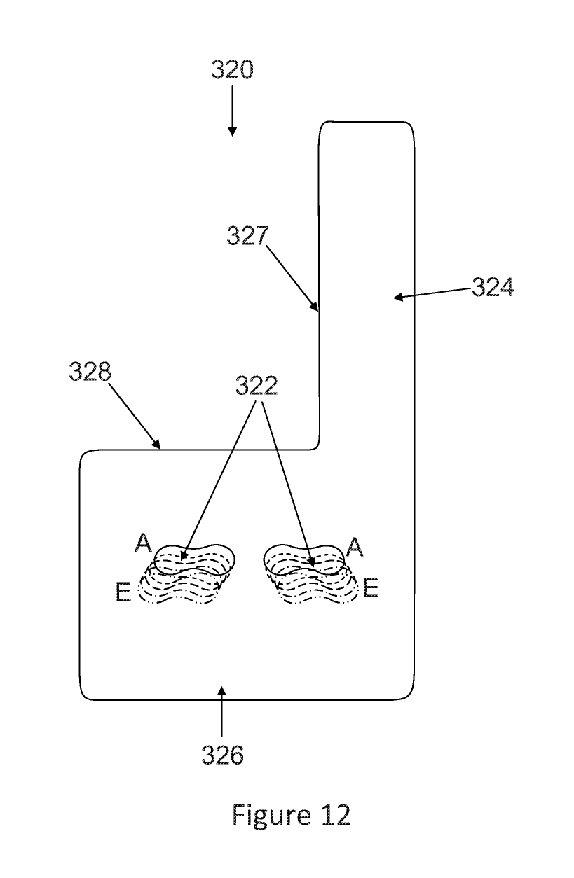

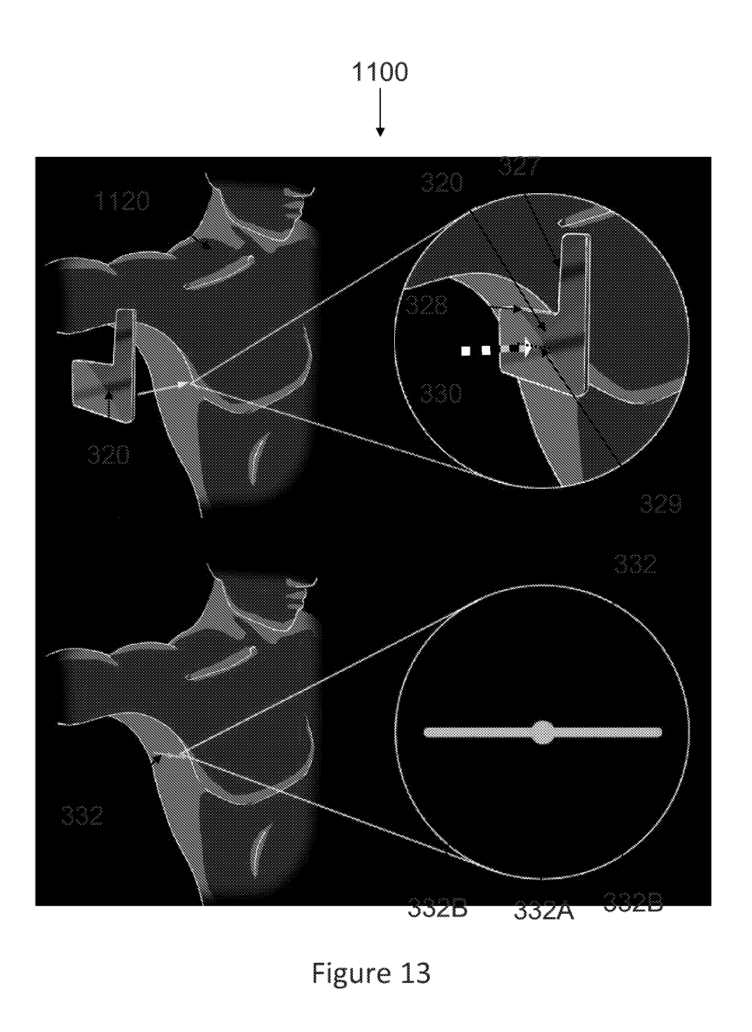

FIGS. 12-13 schematically illustrate two embodiments of placement accessories. FIG. 12 illustrates an embodiment of a placement accessory adjustable to a patient's size, while FIG. 13 illustrates a preferred embodiment, with a fixed-size placement accessory.

Placement accessories (320) are preferably disposable. In some embodiments, one placement accessory (320) is used for both sides of the body. In other embodiments, different placement accessories (320) are used for different parts of the body.

In preferred embodiments, a marking device is used to mark the body to indicate the location of the electrodes, as described hereinbelow.

In preferred embodiments, the marking device (330) is supplied as part of a kit that comprises the placement accessory (320) or placement accessories, the electrodes (110) and the leads (120). In other embodiments, a kit comprises the placement accessory (320) or placement accessories, the electrodes (110) and the leads (120), but not the anthropometer, which is reusable.

FIG. 12 shows an embodiment of a placement accessory (320) in which the placement accessory is adjustable to the patient's size. This adjustable placement accessory is shaped like an “L” with a thick base (324); the ovoid lines (322) are selected according to the size of the patient.

The adjustable placement accessory (320) is placed in position on the patient's body. For a typical adjustable placement accessory, the back side of the adjustable placement accessory (320) is placed against the side of the patient's thorax with the top edge (328) of the base (326) in contact with the armpit, while the inner edge (327) of the vertical portion (324) is in contact with the body, along a vertical line approximately in front of the shoulder joint.

Using the adjustable placement accessory (320) as a guide, the positions in which the electrodes are to be placed is marked on the patient's skin, using an appropriate marking device (330).

In the exemplary adjustable embodiment of FIG. 12, the electrode placement positions are indicated by the ovoids (322), and the user marks the patient's skin along the appropriate ovoid lines (322); the larger the patient, the further down the placement accessory and the further apart the pair of ovoids used. In the exemplary adjustable embodiment of FIG. 12, electrode placement (and marking lines) for the smallest patients is indicated by the solid lines (322A), while electrode placement for the largest patients is indicated by the dash-double dot lines (322E).

FIG. 13A-D shows a preferred embodiment of a fixed-size placement accessory and methods of creating the marks on the patient's skin surface. In the fixed-size embodiment of FIGS. 13A-D, the electrodes are in a fixed position relative to the armpit. The placement accessory (320) is placed against the patient's side (FIG. 13A), as described above, with the top edge of the base (328) in contact with the armpit, while the inner edge (327) of the vertical portion is in contact with the body, along a vertical line approximately in front of the shoulder joint.

The shape of the placement accessory ensures consistent and repeatable marking of the electrodes' position, both for repeated applications of electrodes to the same subject and for application of electrodes between subjects. The L-shape of the placement accessory ensures that, in a preferred embodiment, for any subject, the electrodes are positioned a predetermined distance below the armpit and a point halfway between paired electrodes is a predetermined distance behind the front plane of the shoulder. In preferred embodiments, the point halfway between paired electrodes is the center of an alignment mark.

In some embodiments, the placement accessory comprises a gap or slit (329), and the marker (330, broken arrow) is swiped across the placement accessory (320) thereby marking the patient's skin. In other embodiments, the placement accessory comprises markings, such as, but not limited to, temporary tattoos or stickers, on the side facing the patient's skin. Swiping the marker (330, broken arrow) removes the markings from the back of the placement accessory and adheres them to the patient's skin.

The goal of the skin marking is to serve as a target for very accurate location of the paired electrodes. Moreover, the mark can remain on the skin even if an electrode pair is removed—when an electrode is removed and repositioned or a new electrode pair is used to replace an old or defective electrode pair, the new pair will be in a position identical to that of the old pair.

The mark (332) on the patient's skin is shown in FIG. 13C, with an enlarged view in FIG. 13D. The center (332A) of the mark differs from the remainder of the mark (332B) to assist in proper alignment of the electrode pair (110A), as described hereinbelow.

The mechanism marking the skin can be a pen, a pencil, a marking pen, an IR laser marker, a temporary tattoo, a sticker, a frangible ink cartridge and any combination thereof. The mechanism can be separate from the placement accessory or it can be a part of the placement accessory, or it can be attached to the placement accessory. Temporary tattoos and stickers are typically attached to the placement accessory, pens, pencils, marking pens and laser markers are typically separate from it, and a frangible ink cartridges can be a integral part of the placement accessory.

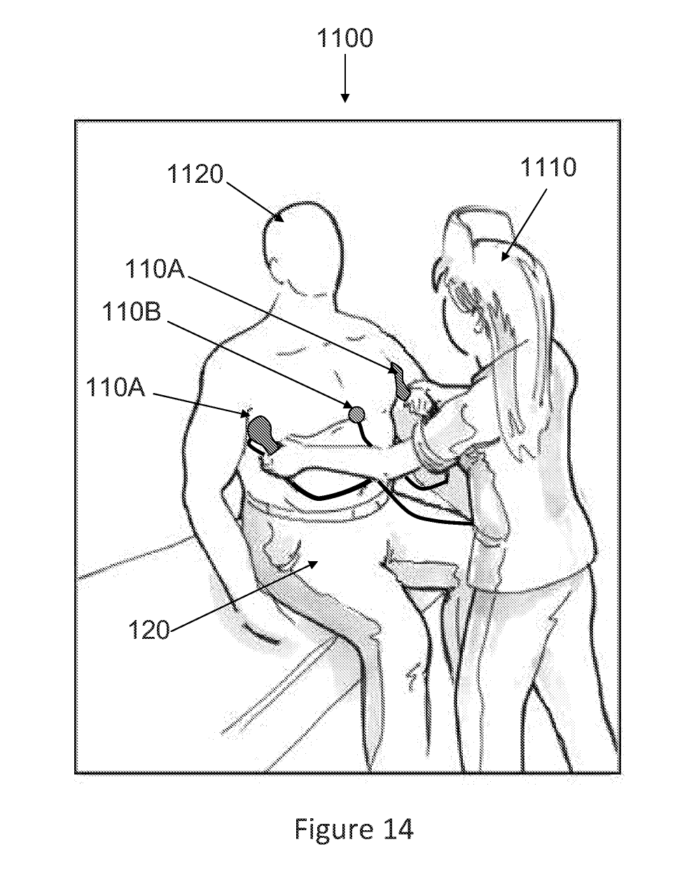

The electrodes (110) are then placed on the body, as illustrated in FIG. 14, which shows (1100) a user (1110) applying the electrodes (110A, 110B) to the skin of a subject (1120). The paired electrodes (110A) are applied at the positions indicated by the marks on the skin.

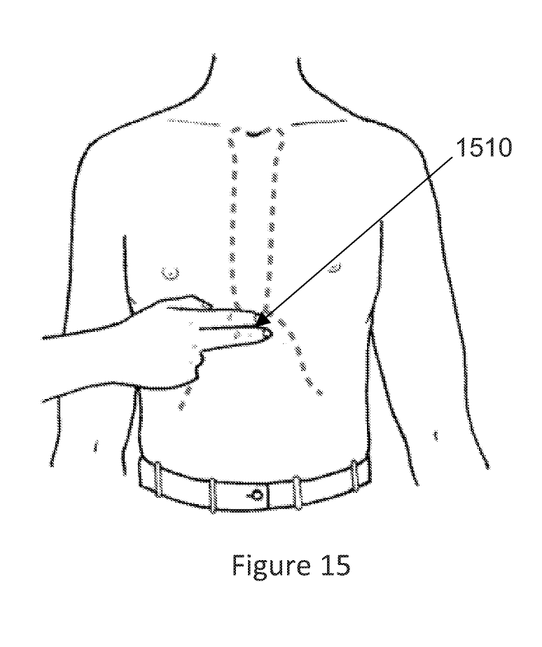

FIG. 15 shows the method of placement for the unpaired electrode in a typical embodiment with five electrodes. In such embodiments, the single electrode) is applied to base of the sternum. The fingers are used to find, by feel, the Xiphoid process (1510) at the bottom of the sternum. The unpaired electrode (110B) is then placed on the Xiphoid process.

In some embodiments, the electrodes are equally spaced, while, in others, the spacing is unequal. The placement can be at defined angular positions around the chest with respect to a predetermined fixed point, or the placement can be symmetrical with respect to the sagittal plane so that left-side placement is the same as right side placement, or the placement can be symmetrical with respect to the coronal plane, so that the placement on the front of the patient is the same as the placement on the back of the patient.

The electrodes are attached to the leads (120) and the leads (120) to the monitoring unit (200). Attachment of the leads (120) to the monitoring unit (200) and application of the electrodes (120) to the body can be done in any order; the order used will be that most likely to provide a trouble-free application and assembly of the unit and produce accurate results.

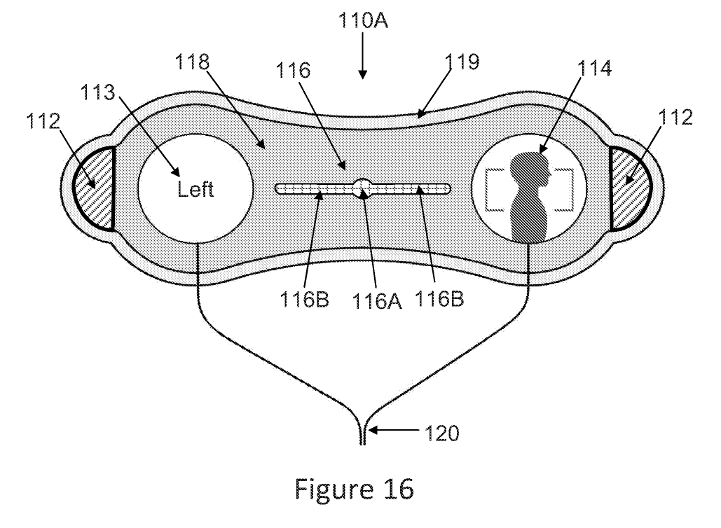

FIG. 16 shows an embodiment of paired electrodes (110). The electrode pair comprises, preferably, a pair of handles (112), although more or fewer handles can be used. The handles (112) are used to hold the electrodes during placement. The electrodes further comprise an indicator (114), in this case, the silhouette of a person, to indicate the end of the electrodes adapted to be towards the front of the chest. Further indication of the direction of placement and of the side of the body on which the electrode pair is to be placed is provided by the text (113). The text can indicate side and direction by words, such as, for non-limiting example, “Left, Back”, or the user can be given instructions, such as, for non-limiting example, “place each electrode pair such that the words are at the rear and the silhouette faces towards the sternum”. In addition to or in place of the above, any other means known in the art can be used for ensuring that each electrode pair is placed on the correct side of the body with the correct electrode of the pair at the rear and the correct electrode of the pair at the front.

The electrode carrier (119) comprises a sticky electrode tape (118) on its reverse side, for adhering the electrode to the patient.

The body of the electrode also comprises an alignment window (116) such that the accuracy can be determined of the placement of the electrode in reference to a marker on the patient's thoracic region. In preferred embodiments, the alignment window (116) is transparent or translucent, or is formed by markings on a transparent or translucent backing although the alignment window (116) can be formed by a gap in the electrode carrier. If a transparent or translucent region is used, the region must be substantially transparent, such that the marking on the subject's skin can be seen easily.

In preferred embodiments, the alignment window comprises a central portion (116A), preferably circular, and an alignment line (116B), preferably extending frontward and backward from the central portion (116A). The central portion (116A) enables accurate alignment of the center of the electrode pair (110A), while the alignment line (116B) enables accurate up-down alignment, as described hereinbelow.

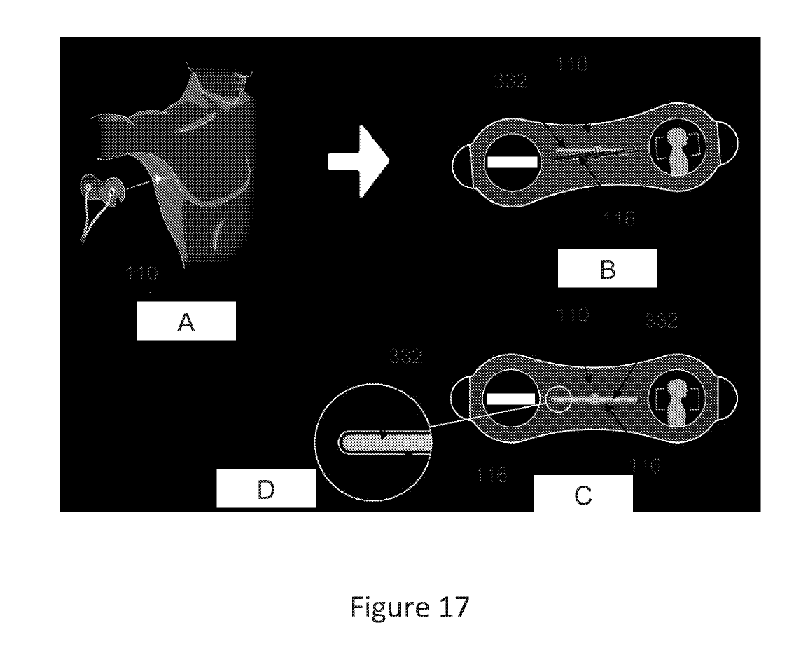

FIG. 17A-D shows an embodiment of a method of placing the electrodes:

The electrode pair contains an alignment window (116) with the same shape as the skin mark (332). The skin mark functions as a target for the alignment window.

Step 1—When bringing the electrodes close to the skin (FIG. 17A), match the circular portion of the target (FIG. 13, 332A) in the center of the skin mark, to the circular central portion (FIG. 16, 116A) of the alignment window.

Step 2—Match the alignment line (FIG. 16, 116B) of the electrode's alignment window (116) to correspond with the target alignment line (FIG. 13, 332B). FIG. 17B illustrates an incorrect position, whereas FIG. 17C illustrates the correct position and FIG. 17D illustrates a close-up of the correct position. The close-up shows the left end (circled) of the alignment window (116) and alignment mark (332).

Combining the 2 steps above ensures the front-back position (thanks to step 1) and up/down balance (thanks to step 2).

In summary:
1. A set of five electrodes is provided, comprising two pairs of electrodes and one unpaired electrode.
2. The two paired electrodes go under the two armpits, one under each armpit, while the unpaired electrode is placed on the sternum
3. The electrodes are suitable for monitoring session of up to few days. Therefore, the materials of which they are comprised, such as the gel, adhesives and backing materials are selected such that they support long term conductance performance, provide integrity of the gel over the entire time of use, and are biocompatible.
4. The electrodes in the set are all pre-wired to a single connector for easy connection and disconnection during the monitoring period of up to a few days
5. Graphic visual indicators are used to aid positioning during placement of the underarm electrodes. The indicators comprise:
   a. Text (113) and silhouette (114) ensure that the correct electrode pair is placed under the correct armpit (left electrode to left armpit, right electrode to right armpit)
   b. The side on which the text (113) and silhouette (114) are printed ensures that the correct (gel) side is placed against the body
   c. The direction of the text (113) and silhouette (114) ensures that the front-back and top-bottom direction of the electrode pairs is correct. For example, they ensure that the front-left electrode is towards the sternum on the left side of the body, while the back-left electrode is towards the back on the left side of the body
   d. The alignment window (116) helps ensure that the electrode pairs are accurately positioned with respect to the armpits and the shoulder joints, that they are the predetermined distance down from the armpits and the predetermined distance back from the shoulder joints, and that the centers of the electrodes are level with each other.

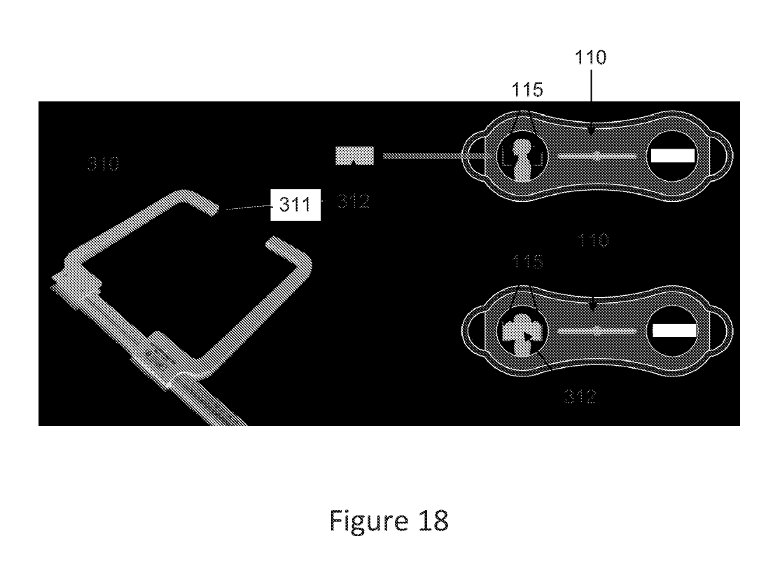

FIG. 18A-C shows use of a second marker to enable accurate placement of the anthropometer (310) on the patient's thoracic region for accurate patient size calibration (discussed hereinbelow). FIG. 18A shows an anthropometer, where the anthropometer tip (311) has a known shape, size and area (312). After placement of the electrodes, the patient's size is measured for normalization. For measurement, each tip (115) of the anthropometer is placed on one of the covers (110) of the paired electrodes, one tip on the cover of the electrode pair on the right side of the body and the other tip on the cover of the electrode pair on the left side of the body. As shown in FIG. 18B, the position for each tip is shown by an indicator (115) that delineates the correct position for placing the anthropometer tip (115).

FIG. 18C shows the caliper tip (312) in the correct position over the anthropometer locator (115).

It should be noted that the cardiac rate can be measured via an ECG, and that blood pressure can be measured via any conventional blood pressure measurement means. Such means include, but re not limited to, a sphygmomanometer, an arterial catheter system, via pulse wave velocity (PWV) measurement systems, via an ambulatory blood pressure monitoring system, or via any other means known in the art.

According to one embodiment, the system can be calibrated according to one of the following methods:
1. The measured voltage can be calibrated with respect to the size of the subject.
2. The measured voltage can be calibrated with respect to the breathing cycle of the subject.
   The calibration can be over a predetermined phase in a breathing cycle (either end of inhalation or end of exhalation)
   The calibration can be over a single inhalation/exhalation cycle.
   The calibration can be over a plurality of inhalation/exhalation cycles.
      Calibration with Respect to Subject Size

In order to improve the accuracy of the measurements, the controller can be configured to calibrate the measured voltage with respect to the size of the patient.

The size of the patient can be defined in terms of the thoracic region by its width, depth (front-to-back), circumference, perimeter length, diameter, radius, length of an axis, cross-sectional area, surface area, volume, or any combination thereof.

It has been found that, in many cases, for patients with the same resistivity, the relationship between patient size and measured voltage is linear so that the calibrated voltage V_{c} can be determined according to the formula V_{c}=Vₘ−a(Pₘ−P_{c}), where a is a constant, Pₘ is the measured patient size and P_{c} is a standard cross-section size.

Calibration with Respect to Breathing Cycle

The resistance of the human thoracic region will change as the patient breathes. At different phases of the breathing cycle there are different amounts of air in the lungs. Since air is an electrical insulator, at points in the breathing cycle where there is more air in the lungs, such as at the end of an inhalation, the resistance of the thorax will be greater than at points in the breathing cycle, such as the end of an exhalation, when there is less air in the lungs. Calibration of the measured voltage can therefore be done either within a single breathing cycle or over a plurality of breathing cycles.

Calibration with Respect to a Single Breathing Cycle

If the calibration is with respect to a single breathing cycle, the calibration can be performed by, for each calibrated voltage difference, (a) taking a plurality of voltage difference measurements over a period of time encompassing a single breathing cycle, a single inhalation/exhalation event; and (b) calculating the calibrated voltage difference V_{c} from the average of the plurality of measurements over the single breathing cycle, so that, for example, the calibrated voltage difference V_{c} can be calculated as the mean of the voltage differences, the median of the voltage differences, or the mode of the voltage differences. In preferred embodiments, the calibrated voltage difference V_{c} is the median of the voltage differences, such that half of the measured voltage differences are greater than the median and half are less than the median,

If the calibrated voltage difference V_{c} is calculated from the median of the voltage differences, then V_{c} is calculated from

$${V}_{c} = \frac{1}{N} {\sum }_{i = 1}^{N} {V}_{m i}$$

where Vₘᵢ is the measured voltage difference and there are N voltage difference measurements per breathing cycle.

Calibration with Respect to a Plurality of Breathing Cycles

If the calibration is with respect to a plurality of breathing cycles, the calibration can be performed by, for each calibrated voltage difference, (a) taking a plurality of voltage difference measurements for each breathing cycle, each inhalation/exhalation event, over a period of time encompassing a plurality of breathing cycles; (b) finding the minimum voltage difference for each breathing cycle, and (c) averaging the plurality of minimum voltages s difference so that, for example, the calibrated voltage difference V_{c} can be calculated from the mean of the voltage differences V_{min,i} as

$${V}_{c} = \frac{1}{N} {\sum }_{i = 1}^{N} {V}_{min , i}$$

where V_{min,i} is the minimum measured voltage difference per breathing cycle and there are N breathing cycles per calibrated voltage difference.

In preferred embodiments, the calibrated voltage difference V_{c} is the median of the voltage differences V_{min,i}, such that half of the measured voltage differences are greater than the median and half are less than the median.

1-116. (canceled)

117. A patient monitoring system comprising:
a. at least one first pair of electrodes wired to each other, adapted to pass known current through said patient's chest;
b. a voltage measuring unit capable of measuring a voltage difference between at least one second pair of electrodes when said first pair of electrodes is passing said known current through said portion of said patient through said first pair of electrodes, when said first and said second pair of electrodes are in contact with said patient;
c. a controller capable of determining at least one biological parameter in at least one portion of said patient's chest, using one or more said voltage differences measured between said at least one second pair of the electrodes; and
d. a patient measurement system;
wherein said patient measurement system is adapted to ensure correct placement of said at least one first pair of electrodes and said at least one second pair of electrodes on said patient's chest, such that said at least one first pair of electrodes and said at least one second pair of electrodes are consistently placeable in the same position on said patient's chest.

118. The system of claim 117, wherein at least one of the following is being held true (a) electrical contact of said electrodes with said patient is provided by a member of a group consisting of: at least one of said electrodes is operable to contact the skin surface of a patient, at least one of said electrodes is implantable in said patient, and any combination thereof; (b) said electrodes are either equally spaced on said patient's chest, or unequally spaced on said patient's chest; (c) the electrodes are placed in positions selected from a group consisting of: at predetermined fractions of the circumference, starting from at least one predetermined point; at predetermined distances from at least one starting point; and any combination thereof; further wherein said predetermined starting point is selected from a group consisting of: the sternum, the front of the armpit, the back of the armpit, the backbone, and any combination thereof; (d) said electrodes are placed in a manner selected from a group consisting of: selecting a fixed point on said patient's chest and placing the electrodes at defined angular positions from said fixed point around said chest; placing the electrodes in an equally spaced manner, placing the electrodes symmetrically with respect to the sagittal plane of said chest; and placing the electrodes symmetrically with respect to the coronal plane of said chest; and any combination thereof; (e) said patient's chest is said patient's thorax; (f) said biological parameter is selected from a group consisting of resistance, derived resistivity, conductance, derived conductivity, derived lung fluid volume, cardiac rate, ECG, respiratory rate, respiratory pattern, blood pressure, and any combination thereof; further wherein said lung fluid volume is either determined from said conductivity; or said lung fluid is extracellular fluid; (g) any combination thereof.

119. The system of claim 117, wherein at least one of the following is being held true (i) said monitoring system is configured for a monitoring procedure selected from a group consisting of electrocardiography and body surface mapping; (iii) said system is configured to monitor at least one selected from a group consisting of: bio-impedance; impedance cardiography; intra-thoracic impedance, phase-delay measurement and any combination thereof; (iii) the system is configured for a monitoring procedure selected from a group consisting of: (a) electrical impedance tomography (EIT), wherein said voltage measuring instrument is configured to measure the voltage in a plurality of pairs of electrodes and said controller is configured to calculate said biological parameter based on said measured voltages, said biological parameter being conductivity in a plurality of voxels in the thorax; and (b) parametric EIT (pEIT), wherein said voltage measuring instrument is configured to measure voltages in a plurality of pairs of electrodes and said controller is configured to calculate said biological parameter based on said measured voltages, said biological parameter being at least one of a group consisting of conductivity, resistivity, conductance and resistance of at least one organ in the thorax.

120. The system of claim 117, wherein at least one of the following is being held true (a) said system additionally comprises means adapted to deliver at least one drug to said patient; (b) said system further comprises a screen adapted to display at least one said at least one biological parameter; (c) said controller further comprises a database adapted to store at least one said at least one biological parameter, said system further comprises means to enable a user to enter a comment, said comment storable in said database; said means to enable a user to enter a comment is selected from a group consisting of a keyboard, a touchscreen, voice commands and any combination thereof; (c) said electrodes are either commercially available electrodes or proprietary electrodes; (d) any combination thereof.

121. The system of claim 119, wherein at least one of the following is being held true (a) said drug delivery means is a computer-controllable drug delivery device, wherein the drug is delivered by a method selected from a group consisting of injection, providing pills and providing a drinkable liquid; (b) said drug delivery means is adapted to titrate said at least one drug to said patient, such that said system is adapted to provide closed-loop monitoring of said patient; and any combination thereof.

122. The system of claim 117, wherein the size of said patient's thoracic region is selected from a group consisting of: its width, its depth (front-to-back), its circumference, its perimeter length, its diameter, its radius, the length of an axis, its cross-sectional area, its surface area, its volume, and any combination thereof and any combination thereof; further wherein said patient measurement system comprises at least one mechanism for measuring said size of at least a portion of said thoracic region; wherein said mechanism for measuring the size of at least a portion of said thoracic region is selected from a group consisting of an anthropometer, a measuring tape and any combination thereof.

123. The system of claim 117, wherein said patient measurement system comprises at least one mechanism for reproducibly specifying at least one position on said thoracic region; further wherein said mechanism for reproducibly specifying at least one position on said thoracic region comprises a placement accessory, said placement accessory adapted to ergonomically guide correct positioning of said electrodes and thereby to enable consistent and repeatable placement of said electrodes at a predetermined position on said patient's thoracic region; wherein said placement accessory is L-shaped.

124. The system of claim 123, wherein said placement accessory is disposable.

125. The system of claim 123, wherein said placement accessory comprises a slit of predetermined shape, said slit adapted to enable the surface of said patient's thoracic region to be marked.

126. The system of claim 117, wherein said patient measurement system comprises at least one mechanism for marking on the surface of said thoracic region in at least one predetermined position; further wherein said mechanism for marking on the surface of said thoracic region comprises a pen, a pencil, a marking pen, an IR laser marker, a temporary tattoo, a sticker, a frangible ink cartridge and any combination thereof.

127. The system of claim 126, wherein at least one of the following is being held true (a) at least one of said temporary tattoo, said sticker and said frangible ink cartridge is mounted on said placement accessory; (b) said placement accessory comprises said mechanism for marking on the surface of said thoracic region; and any combination thereof.

128. The system of claim 117, wherein at least one of the following is being held true (a) said system further comprising a power supply adapted to power said screen, said controller, and to provide said current to said electrodes; (b) said measurement system is comprised in a first kit adapted to be provided as a unit; (c) wherein a second kit adapted to be provided as a unit comprises at least one of a group consisting of: said mechanism for measuring said size of at least a portion of said chest, said mechanism for reproducibly specifying at least one position on said chest, and said mechanism for marking on the surface of said chest each said at least one position; (d) said biological parameter is determined from a calibrated voltage difference;

129. The system of claim 128, wherein at least one of the following is being held true (a) said calibration is selected from a group consisting of: calibration to the size of the patient, calibration to the patient's breathing cycle, and any combination thereof; (b) said size calibration is a linear calibration; (c) said size calibration is calculated from V_{c}=Vₘ−a(Pₘ−P_{c}), where a is a predetermined constant, Pₘ is the measured patient size and P_{c} is a predetermined standard cross-section size; (d) said measured patient size is selected from a group consisting of: width of a predetermined portion of the body, circumference of a predetermined portion of the body, area of a predetermined portion of the body, thickness of a predetermined portion of the body, and any combination thereof; (e) said portion of the body is selected from a group consisting of: a predetermined portion of the thorax, a predetermined portion of the height, a predetermined portion of the abdomen, and any combination thereof; (f) said calibration to the patient's breathing cycle is selected from a group consisting of: calibration to a single breathing cycle, calibration to a predetermined portion of a breathing cycle, calibration to a plurality of breathing cycles and any combination thereof; (g) said calibration to said single breathing cycle consists of averaging a plurality of voltage difference measurements, said voltage difference measurements taken during a single breathing cycle; (h) said average of said voltage difference measurements for a single breathing cycle is selected from a group consisting of: the mean of the voltage difference measurements, the median of the voltage difference measurements and the mode of the voltage difference measurements; (i) and any combination thereof.

130. The system of claim 129, wherein at least one of the following is being held true (a) said predetermined portion of a breathing cycle is selected from a group consisting of: at least a portion of an inspiration, at least a portion of an exhalation, the beginning of an inspiration, the beginning of an exhalation, the end of an inspiration, the end of an exhalation, and any combination thereof; (b) said calibration to said predetermined portion of a breathing cycle consists of averaging a plurality of voltage difference measurements, said voltage difference measurements taken either during a single breathing cycle or during a plurality of breathing cycles; (c) said average of said voltage difference measurements for said predetermined portion of a breathing cycle is selected from a group consisting of: the mean of the voltage difference measurements, the median of the voltage difference measurements and the mode of the voltage difference measurements; (d) said calibration to said plurality of breathing cycles consists of averaging a plurality of voltage difference measurements, each said voltage difference measurement being the minimum voltage difference measured during a single breathing cycle; (e) said average of said voltage difference measurements for a plurality of breathing cycles is selected from a group consisting of: the mean of the voltage difference measurements, the median of the voltage difference measurements and the mode of the voltage difference measurements; and any combination thereof.

131. A method for monitoring a patient comprising steps of:
a. providing a system for monitoring the lung fluid volume of patients, comprising:
i. at least one first pair of electrodes wired to each other, adapted to pass known current through said patient's chest;
ii. a voltage measuring unit capable of measuring a voltage difference between at least one second pair of electrodes when said first pair of electrodes is passing said known current through said portion of said patient through said first pair of electrodes, when said first and said second pair of electrodes are in contact with said patient;
iii. a controller capable of determining at least one biological parameter in at least one portion of said patient's chest, using one or more said voltage differences measured between said at least one second pair of the electrodes; and
iv. a patient measurement system;
b. measuring said patient with said patient measurement system;
c. placing said electrodes in electrical contact with said patient as indicated by said patient measurement system, thereby ensuring correct placement of said at least one first pair of electrodes and said at least one second pair of electrodes on said patient's chest, such that said at least one first pair of electrodes and said at least one second pair of electrodes are consistently placeable in the same position on said patient's chest;
d. measuring, with said voltage measuring unit, at least one voltage difference across at least one pair of said at least one second pair of electrodes;
e. determining, with said controller, using at least one of said at least one voltage differences, at least one of said at least one biological parameters in at least one portion of said patient's chest.

132. The method of claim 131, wherein at least one of the following is being held true (a) said steps of placing said electrodes in electrical contact with said patient are selected from a group consisting of: steps of contacting the skin surface of a patient with at least one said electrode, steps of using at least one said electrode implanted in said patient, and any combination thereof; (b) said method additionally comprising steps of spacing said electrodes either equally spaced on said patient's chest, or unequally spaced on said patient's chest; (c) said method additionally comprising steps of placing the electrodes in positions selected from a group consisting of: at predetermined fractions of the circumference, starting from at least one predetermined point; at predetermined distances from at least one starting point; and any combination thereof; (d) said method additionally comprising steps of selecting said predetermined starting point is selected from a group consisting of: the sternum, the front of the armpit, the back of the armpit, the backbone, and any combination thereof; (e) said method additionally comprising steps of placing said electrodes on said patient's chest in a manner selected from a group consisting of: selecting a fixed point on said patient's chest and placing the electrodes at defined angular positions from said fixed point around said chest; placing the electrodes in an equally spaced manner; placing the electrodes symmetrically with respect to the sagittal plane of said chest; placing the electrodes symmetrically with respect to the coronal plane of said chest; and any combination thereof; (f) said method additionally comprising steps of selecting said biological parameter from a group consisting of resistance, derived resistivity, conductance, derived conductivity, derived lung fluid volume, cardiac rate, ECG, respiratory rate, respiratory pattern, blood pressure, and any combination thereof; (g) said method additionally comprising steps of determining said lung fluid volume from said conductivity; (h) and any combination thereof.

133. The method of claim 131, additionally comprising at least one step selected from a group consisting of (i) configuring said monitoring system for a monitoring procedure selected from a group consisting of electrocardiography and body surface mapping; (ii) configuring said system to monitor at least one selected from a group consisting of: bio-impedance; impedance cardiography; intra-thoracic impedance, phase-delay measurement and any combination thereof; (iii) configuring said system for a monitoring procedure selected from a group consisting of: (a) electrical impedance tomography (EIT), wherein said voltage measuring instrument is configured to measure the voltage in a plurality of pairs of electrodes and said controller is configured to calculate said biological parameter based on said measured voltages, said biological parameter being conductivity in a plurality of voxels in the thorax; and (b) parametric EIT (pEIT), wherein said voltage measuring instrument is configured to measure voltages in a plurality of pairs of electrodes and said controller is configured to calculate said biological parameter based on said measured voltages, said biological parameter being at least one of a group consisting of conductivity, resistivity, conductance and resistance of at least one organ in the thorax.

134. The method of claim 131, additionally comprising at least one step selected from a group consisting of (a) selecting said organ from a group consisting of a heart and a lung; (b) providing said system with means adapted to deliver at least one drug to said patient; (c) providing said drug delivery means as a computer-controllable drug delivery device, wherein the drug is delivered by a method selected from a group consisting of injection, providing pills and providing a drinkable liquid; (d) providing closed-loop monitoring of said patient by using said drug delivery means to titrate said at least one drug to said patient; (e) providing a screen adapted to display at least one said at least one biological parameter; (f) providing said controller with a database adapted to store at least one said at least one biological parameter, (g) providing means enabling a user to enter a comment, said comment storable in said database; (h) selecting said means to enable a user to enter a comment from a group consisting of a keyboard, a touchscreen, voice commands and any combination thereof; (i) and any combination thereof.

135. The method of claim 131, additionally comprising at least one step selected from a group consisting of (a) displaying said comment on said display unit; (b) for each said comment, displaying on said screen at least one indication that said comment has been entered into said system; (c) indicating on said screen, for each said comment, the time each said comment was entered; (d) providing said screen as a touchscreen; (e) providing said electrodes as either commercially available electrodes or proprietary electrodes; (f) determining the size of said patient's thoracic region by measuring at least one selected from a group consisting of: its width, its depth (front-to-back), its circumference, its perimeter length, its diameter, its radius, the length of an axis, its cross-sectional area, its surface area, its volume, and any combination thereof and any combination thereof; (g) providing at least one mechanism for measuring said size of at least a portion of said thoracic region; (h) selecting said mechanism for measuring the size of at least a portion of said chest from a group consisting of an anthropometer, a measuring tape and any combination thereof; (i) providing at least one mechanism for reproducibly specifying at least one position on said thoracic region; (j) and any combination thereof.

136. The method of claim 131, additionally comprising at least one step selected from a group consisting of (a) providing said mechanism for reproducibly specifying at least one position on said thoracic region as a placement accessory, said placement accessory adapted to ergonomically guide correct positioning of said electrodes and thereby enabled to consistently and repeatably place said electrodes at a predetermined position on said patient's thoracic region; (b) providing an L-shaped placement accessory; (c) providing said placement accessory comprising a slit of predetermined shape, thereby enabling marking of the surface of said patient's thoracic region; (d) providing at least one mechanism for marking on the surface of said thoracic region in at least one predetermined position; (e) selecting said mechanism for marking on the surface of said thoracic region from a group consisting of: a pen, a pencil, a marking pen, an IR laser marker, a temporary tattoo, a sticker, a frangible ink cartridge and any combination thereof; (f) mounting at least one of said temporary tattoo, said sticker and said frangible ink cartridge on said placement accessory; (g) comprising said placement accessory as part of said mechanism for marking on the surface of said thoracic region; (h) and any combination thereof.

137. The system of claim 117, wherein said display unit (200) additionally comprises a means of holding at least one of said electrode leads (120) and said electrodes (110); wherein said holding means comprises a member of a group consisting of: a recess in said display unit (200), a slot through said display unit (200), a clip, an elastic band, a strap, a buckle, and any combination thereof.
